(19) United States
(12) Patent Application Publication
(10) Pub. No.: US 2026/0053866 A1
(43) Pub. Date: Feb. 26, 2026

(54) COMPOSITIONS FOR USE IN TREATING AND/OR PREVENTING TYPE 2 DIABETES, PRE-DIABETES, AND/OR A SYMPTOM THEREOF

(71) Applicant: De Faire Medical AB, Uppsala (SE)

(72) Inventor: Johan De Faire, Uppsala (SE)

(21) Appl. No.: 19/104,898

(22) PCT Filed: Aug. 17, 2023
(86) PCT No.: PCT/EP2023/072746

§ 371 Date: Feb. 19, 2025

(30) Foreign Application Priority Data
Aug. 19, 2022 (GB) 2212123.0

(51) Int. Cl. A61K 35/742; A61K 9/48; A61K 35/00; A61K 36/899; A61P 3/10

(52) U.S. Cl. CPC A61K 35/742; A61K 9/4891; A61K 36/899; A61P 3/10; A61K 2035/115; A61K 2236/19

(57) ABSTRACT

The present invention provides methods of using a composition comprising one or more bacterial species of the Bacillus genus, rice bran, L-cysteine and a high molecular weight low osmolality carbohydrate for the treatment and/or prevention of type 2 diabetes, pre-diabetes, and/or a symptom thereof in a subject. Further uses of the composition are also provided herein.

The present invention provides methods of using a composition comprising one or more bacterial species of the Bacillus genus, rice bran, L-cysteine and a high molecular weight low osmolality carbohydrate for the treatment and/or prevention of type 2 diabetes, pre-diabetes, and/or a symptom thereof in a subject. Further uses of the composition are also provided herein.

### BACKGROUND

There is increasing evidence that gut microbiome is involved in the development of human diseases, including but not limited to disorders of immune system, energy metabolism, lipid metabolism, and glucose metabolism [1, 2]. Appropriate development of the metabolic functionality of the human body is strongly driven by this symbiosis between intestinal microbiome and the host organism. The amounts of microorganisms in a human-being is estimated to be around 3.8×10¹³, with a total mass between 200 g to 1 kg of the total body weight of a person [3-5]. A wide range of biological processes are influenced by the gut microbiome, such as energy balance homeostasis and digestion of nutrients. The trigger for this function is the production of short-chain fatty acids (butyrate, propionate, and acetate), which are considered as signalling molecules in gut and extraintestinal tissues [6]. Other functions are participation in the transport and metabolism of carbohydrates and amino acids, the production of fat-soluble and water-soluble vitamins [7], the elimination of xenobiotics and drugs, and in bile acid metabolism [8]. Disturbances in composition and subsequently also in the functionality of the host-microbiome interaction can result in a chronic systemic inflammation induced by bacterial fragments, which appear in the circulation as consequence of a malfunctioning gut barrier. Hence, a malfunctioning gut barrier, has considerable trigger effects for host adiposity and insulin resistance. [9].

Recent evidence suggests that there are certain bacterial species that may interact with host metabolism, e.g. through metabolite-mediated stimulation of enteric hormones. Modulation of the gut microbiome by means of external supplementation with bacteria and probiotics may offer an interesting opportunity for supplementary treatment of several diseases [10]. By definition probiotics are substances contained in certain foods, which may modulate the composition of gut microbiome, thereby influencing short-chain fatty acid production, satiety, weight control, energy homeostasis, and suppressing the growth of pathogens and immunomodulatory actions [11, 12]. Indeed, probiotics have been shown to be effective in varied clinical conditions—ranging from infantile diarrhoea, necrotizing enterocolitis, antibiotic-associated diarrhea, inflammatory bowel disease to cancer, female urogenital infection, surgical infections, insulin resistance, and oxidative stress [13-15].

Diabetes mellitus is a chronic systemic disease associated with complications in most of the body organs. It is a pandemic, which affects ˜9% of the population in the world [21]. Changes in pancreatic beta-cell function and insulin resistance lead to relative insulin deficiency and impaired cellular response to insulin. A significant symptom of the disease is hyperglycemia, which can cause several severe secondary complications [22, 23]. Anti-diabetic drugs are commonly used in accordance with national and international treatment guidelines [24, 25] and the most commonly used drug, metformin, can have limited effect and can cause adverse effects on the composition of the bacterial microfauna in the intestine tract.

Accordingly, there is a need for a novel composition, supplement and/or therapeutic for use in treating and/or preventing type 2 diabetes and/or a symptom thereof.

### BRIEF SUMMARY OF THE DISCLOSURE

Recently, the gut microbiome was found to be altered in diabetic patients, e.g. with increased ratios of Bacteroides to Clostridium species in the gut, and increased numbers of various opportunistic pathogens [26-29]. Circulating gram-positive gut bacteria were detected in blood samples of diabetic patients [28]. Changes in gut microbiome may lead to metabolic endotoxemia through the release of lipopolysaccharides, consequently stimulating inflammation and insulin resistance [30]. The epithelial enteroendocrine L cells play a role in producing inflammation, and their number was positively or negatively correlated with the abundance of 25 bacterial taxa in the intestine [31, 32].

In light of these findings, the effect of probiotic supplementation for diabetes and diabetes-related complications has been investigated.

The inventors have previously developed a composition (e.g. a nutritional supplement) based on (fermented) rice bran with Bacillus subtilis and Bacillus coagulans, which provide prebiotic and probiotic properties. The inventors have now surprisingly shown that the composition (referred to as AB001 herein) may advantageously be used to safely improve the cardiometabolic situation of a subject, in particular a subject having type 2 diabetes and/or pre-diabetes. For example, the composition may advantageously provide a valuable supplement to existing treatment combinations in a subject having type 2 diabetes and/or pre-diabetes.

The inventors have surprisingly shown AB001 has beneficial effects vs placebo on glycemic control and on biomarkers of inflammation (e.g. chronic systemic inflammation), insulin resistance and ß-cell dysfunction in patients with type 2 diabetes. The inventors have thus advantageously demonstrated that the composition described herein (AB001) may be used to stabilise glucose levels and/or improve glycemic control, in particular in subjects having type 2 diabetes and/or pre-diabetes. Advantageously, the composition described herein may also be used to reduce insulin resistance, ß-cell dysfunction and/or inflammation (e.g. chronic systemic inflammation and/or intestinal inflammation) in subjects having type 2 diabetes and/or pre-diabetes. In some examples, the inflammation may be intestinal and/or pancreatic inflammation.

In addition, the inventors surprisingly observed that in patients with type 2 diabetes, changes in lipid profiles and other parameters of metabolic syndrome were more favourable with AB001 than with placebo. Advantageously, the composition described herein may therefore be used to treat and/or prevent symptoms of metabolic syndrome in subjects having type 2 diabetes and/or pre-diabetes.

AB001 comprises specially selected bacterial strains, which preferably metabolize glucose and other carbohydrates, providing a strong impact on glucose levels and insulin resistance. Metabolised sugars in the intestinal tract, e.g. glucose, from food and beverage are to some extent nutrients to cells of the microbiome (e.g. cells of the gut microbiome) before being resorbed to the blood as energy depots. Not wishing to be bound by theory, it is suggested that AB001 has four glucose-controlling actions—1) Bacilli strains of AB001 and their excreted enzymes act as additional glucose-digesters (in other words, the Bacilli strains of AB001 and their excreted enzymes act in addition to the microbiome (e.g. the gut microbiome) of a subject that was present before consumption of AB001) to further reduce resorption of glucose to the blood, 2) AB001 reduces low-grade pancreatic inflammation making β-cells of the pancreas produce more insulin, and less pro-insulin, 3) cells' sensitivity to insulin is increased by AB001 to a more normalised glucose-insulin mechanism, and 4) AB001 changes the ratio of Low-Density-Lipids, LDL, and High-Density-Lipids, HDL to higher concentration of HDL. The latter means that surplus glucose is converted into blood fats, e.g. triglycerides, to a higher degree which are transported from the blood by HDL to the liver to be degraded. In contrast, LDL does the opposite and transports fats from the liver to the blood. Accordingly, in some instances, AB001 action is driven by the excretion of bioactive substances, such as enzymes, that break down complex organic molecules into nutrients, reactants and energy. In some instances, (increased) glucose levels are normalised due to the presence of more insulin.

It is suggested that, upon resuscitation in the intestinal tract, the Bacillus spp. of the composition for use according to the present invention (and their endospores) scan the biochemical conditions of their micro-environment and start to excrete a unique selection of bio-active substances to optimise the conditions, e.g. pH, conductivity, electrolytes, for their survival and multiplication. Nutrients and substrates are essential for survival and subsequent multiplication. When carbohydrates (in particular glucose) is present in the micro-environment, carbohydrate (e.g. glucose)-targeted enzymes are excreted to break down said substrates.

Advantageously, the compositions for use according to the present invention may be formulated as an acid resistant tablet or capsule. Such formulations are known to resist the acid in the stomach, only to dissolve once reaching the duodenum. The Bacillus spp. of the composition can then be released to settle in the upper part of the intestinal tract where they can reside for about one day before being eliminated from the body through the feces. The bacterial spp. of the composition described herein were selected to preferably and effectively metabolize carbohydrates (in particular glucose). It is suggested that a result of AB001 supplementation is a lower absorption of glucose through the intestine tract. A similar effect and corresponding biochemical findings are seen, when drugs belonging to the class of SGLT-1 inhibitors (α-glucosidase inhibitors, e.g. acarbose or voglibiose) are used in routine diabetes treatment. SGLT-1 inhibitors actively withhold the glucose molecules in the intestine tract, where they finally get metabolized by bacteria populating the large bowel, with methane gas as the final end product. In consequence, patients treated with SGLT-1 inhibitors complained about major problems with flatulence and bloated belly [39, 40]. It is suggested that supplementation with AB001 means that less glucose reaches the blood.

A composition comprising one or more bacterial species of the Bacillus genus and rice bran, for use in treating and/or preventing type 2 diabetes, pre-diabetes, and/or a symptom thereof in a subject is provided herein.

A composition comprising one or more bacterial species of the Bacillus genus, rice bran, L-cysteine and a high molecular weight low osmolality carbohydrate for use in treating and/or preventing type 2 diabetes, pre-diabetes, and/or a symptom thereof in a subject is also provided herein.

A method of treating and/or preventing type 2 diabetes, pre-diabetes, and/or a symptom thereof in a subject is also provided herein, comprising administering a composition comprising one or more bacterial species of the Bacillus genus and rice bran to the subject.

A method of treating and/or preventing type 2 diabetes, pre-diabetes, and/or a symptom thereof in a subject, comprising administering a composition comprising one or more bacterial species of the Bacillus genus, rice bran, L-cysteine and a high molecular weight low osmolality carbohydrate to the subject is also provided herein.

Suitably, the composition may be for treating and/or preventing type 2 diabetes and/or a symptom thereof.

Suitably, administration of the composition to the subject may:
(a) reduce the level of one or more of HbA1c, glucose, insulin, intact pro-insulin and/or hsCRP in the blood;
(b) reduce the HOMA-IR score; and/or
(c) increase the level of adiponectin in the blood.

Suitably, administration of the composition to the subject may control blood glucose.

Suitably, administration of the composition to the subject may degrade glucose.

Suitably, administration of the composition to the subject may metabolise glucose in the gut of the subject, optionally administration of the composition to the subject may metabolise glucose in the intestine of the subject, more optionally administration of the composition to the subject may metabolise glucose in the small intestine of the subject.

Suitably, administration of the composition to the subject may reduce absorption of glucose into the blood of the subject.

Suitably, administration of the composition to the subject may reduce blood glucose concentration in the subject.

Use of a composition comprising one or more bacterial species of the Bacillus genus and rice bran for degrading glucose is provided herein.

Use of a composition comprising one or more bacterial species of the Bacillus genus and rice bran, for:
(a) reducing the level of one or more of the following in the blood of a subject HbA1c, glucose (e.g. fasting glucose), insulin, intact pro-insulin and/or hsCRP;
(b) reducing the HOMA-IR score in a subject; and/or
(c) increasing the level of adiponectin in the blood of a subject, is also provided herein.

Use of a composition comprising one or more bacterial species of the Bacillus genus and rice bran, for controlling blood glucose in a subject is provided herein.

Use of a composition comprising one or more bacterial species of the Bacillus genus, rice bran, L-cysteine and a high molecular weight low osmolality carbohydrate for degrading glucose is also provided herein.

Use of a composition comprising one or more bacterial species of the Bacillus genus, rice bran, L-cysteine and a high molecular weight low osmolality carbohydrate for:
(a) reducing the level of one or more of the following in the blood of a subject HbA1c, glucose, insulin, intact pro-insulin and/or hsCRP;
(b) reducing the HOMA-IR score in a subject; and/or
(c) increasing the level of adiponectin in the blood of a subject is also provided herein.

Use of a composition comprising one or more bacterial species of the Bacillus genus, rice bran, L-cysteine and a high molecular weight low osmolality carbohydrate for controlling blood glucose in a subject is further provided herein.

Suitably, the one or more bacterial species of the Bacillus genus may be selected from B. subtilis and B. coagulans.

Suitably, the composition may comprise B. subtilis and B. coagulans.

Suitably,
a) the B. subtilis species may be selected from the group consisting of: Bacillus subtilis strain DFM 0326 (LMG P-32899) and Bacillus subtilis strain DFM 1015 (LMG P-32900); and/or
c) the B. coagulans species may be Bacillus coagulans strain DFM 0705 (LMG P-32921).

Suitably, the high molecular weight low osmolality carbohydrate may be dextrin.

Suitably, the composition may further comprise one or more bacterial species selected from the group consisting of: Bacillus amyloliquefaciens, Bacillus velezensis, Bacillus sp MT 03, Bacillus atrophaeus, and Pediococcus pentosaceus. Suitably, the composition may further comprise one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis (Dekkera bruxellensis according to old taxonomy), and/or Millerozoma farinosa (Pichia farinosa according to old taxonomy)).

Suitably, the composition may comprise at least about 20% w/w of L-cysteine.

Suitably, the composition may comprise at least about 1×10⁵ of bacteria of the Bacillus genus.

Suitably, the composition may comprise at least about 73% w/w of rice bran.

Suitably, the composition may comprise at least about 0.5% w/w of high molecular weight low osmolality carbohydrate.

Suitably, the composition may further comprise one or more of: vitamin B12, a fatty acid magnesium salt, calcium phosphate, potassium phosphate, silicon dioxide and cellulose, optionally wherein the fatty acid magnesium salt is magnesium stearate.

Suitably, the composition may be formulated as an acid resistant tablet or capsule.

Suitably, the acid resistant tablet or capsule may comprise a film coating, wherein the film coating comprises hydroxypropyl methylcellulose (HPMC).

Suitably, the one or more bacterial species of the Bacillus genus may not be genetically modified.

Suitably, the composition may be for daily administration.

The composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) comprises one or more bacterial species of the Bacillus genus, rice bran, L-cysteine and a high molecular weight low osmolality carbohydrate. Methods of using the composition are also provided herein.

In each of the aspects and embodiments of the invention described herein, rice bran may be replaced with any suitable cereal (e.g. cereal grain) unless the context provides otherwise. In such aspects and embodiments, a suitable cereal may be any suitable cereal bran. Suitable cereals and suitable cereal brans are discussed elsewhere herein.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### Budapest Treaty on the International Recognition of the Deposit of Microorganisms for the Purpose of Patent Procedures

Several strains of bacillus species described in this application were deposited with the Belgian Coordinated Collections of Micro-organisms (BCCM) Laboratorium voor Microbiologie—Bacteriënverzameling (LMG) which is an International Depositary Authority located at Universiteit Gent, K. L. Ledeganckstraat 35, 9000 Gent, Belgium. The deposits were made under the terms of the Budapest Treaty on the International Recognition of the Deposit of Microorganisms for the Purposes of Patent Procedure.

### BRIEF DESCRIPTION OF THE FIGURES

Embodiments of the invention are further described hereinafter with reference to the accompanying drawings, in which:

FIG. 1 shows changes in the Freestyle Libre analysis between the first (week 0 to 2) and the last two weeks (week 5 to 6) of the study observation period.

FIG. 2 shows changes from baseline after 6 weeks of treatment of the measured biomarkers for glycemic control and for the underlying diabetes deteriorations (n=40).

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The invention is based on the surprising finding that a composition (e.g. a nutritional supplement) based on (fermented) rice bran with Bacillus subtilis and Bacillus coagulans may advantageously be used to safely improve the cardiometabolic situation of a subject, in particular a subject having type 2 diabetes and/or pre-diabetes.

The inventors have surprisingly shown AB001 has beneficial effects vs placebo on glycemic control and on biomarkers of inflammation (e.g. chronic systemic inflammation), insulin resistance and ß-cell dysfunction in patients with type 2 diabetes. The inventors have thus advantageously demonstrated that the composition described herein (AB001) may be used to stabilise glucose levels and/or improve glycemic control, in particular in subjects having type 2 diabetes and/or pre-diabetes. Advantageously, the composition described herein may also be used to reduce insulin resistance, ß-cell dysfunction and/or inflammation (e.g. chronic systemic inflammation and/or intestinal inflammation), in particular in subjects having type 2 diabetes and/or pre-diabetes. In some examples, the inflammation may be intestinal and/or pancreatic inflammation. In addition, the inventors surprisingly observed that in patients with type 2 diabetes, changes in lipid profiles and other parameters of metabolic syndrome were more favourable with AB001 than with placebo. Advantageously, the composition described herein may therefore be used to treat and/or prevent symptoms of metabolic syndrome in subjects having type 2 diabetes and/or pre-diabetes.

Accordingly, a composition comprising one or more bacterial species of the Bacillus genus, rice bran, L-cysteine and a high molecular weight low osmolality carbohydrate for use in treating and/or preventing type 2 diabetes, pre-diabetes, and/or a symptom thereof in a subject is provided herein.

Furthermore, a method of treating and/or preventing type 2 diabetes, pre-diabetes, and/or a symptom thereof in a subject, comprising administering a composition comprising one or more bacterial species of the Bacillus genus, rice bran, L-cysteine and a high molecular weight low osmolality carbohydrate to the subject is also provided.

### Compositions for Use According to the Present Invention

The composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject, may be referred to as “MYRKL-S”, “MYRKLS”, “SB001” and/or “AB001”.

AB001 comprises specially selected bacterial strains, which preferably degrade (e.g. metabolize) glucose and other carbohydrates, and also reduce (intestinal and/or pancreatic) inflammation. Not wishing to be bound by theory, it is suggested that AB001 has four glucose-controlling actions, as discussed elsewhere herein.

In the body, complex carbohydrates reaching the small intestine must be hydrolyzed to monosaccharides such as glucose or galactose in order to be transported across the intestinal mucosa. The term carbohydrate is used in particular in biochemistry, where it is a synonym of saccharide. Carbohydrates (saccharides) may be divided into four chemical groupings: monosaccharides, disaccharides, oligosaccharides, and polysaccharides. In general, monosaccharides and disaccharides, which are smaller (lower molecular weight) carbohydrates, are commonly referred to as sugars. A person skilled in the art would readily be able to identify a carbohydrate using methods routine in the art. A non-limiting example of a carbohydrate is glucose. The classical pathway of glucose absorption is across the intestinal brush-border membrane (BBM) and is thought to be predominantly mediated by SGLT1, an apically located sodium-dependent glucose transporter on enterocytes. This symporter (SGLT1) uses the electrochemical gradient of two sodium ions to transport one glucose molecule. Intracellular glucose is released into the interstitial space near the blood capillaries by a mechanism thought to occur by facilitated diffusion via the glucose transporter GLUT2 located in the basolateral membrane of enterocytes.

Glucose is a 6-carbon structure with the chemical formula C6H12O6. It is a ubiquitous source of energy for every organism in the world and is essential to fuel both aerobic and anaerobic cellular respiration.

In some examples, the composition for use according to the present invention may be a glucose degrading composition.

As would be clear to the skilled person, a “glucose degrading composition”, as referred to herein, is a composition which degrades glucose. In other words, a “glucose degrading composition”, as used herein, is a composition which breaks down (e.g. biochemically breaks down) glucose. In the context of the invention, degradation (e.g. breakdown) of a substance (such as a carbohydrate, e.g. glucose) involves converting the substance into one or more other distinct substances. Furthermore, as would be understood by the person skilled in the art, “glucose degrading”, as used herein, refers to the breaking down of glucose and, “glucose degradation”, as used herein, refers to the breakdown of glucose. As would be clear to the skilled person, the composition described herein can be used in vitro or in vivo.

Glucose may be degraded (e.g. broken down) in several different ways. Particularly relevant in the context of the present invention is biochemical degradation. Accordingly, in one example, glucose may be degraded (e.g. broken down) biochemically. As would be known to the skilled person, the biochemical break down of a substance may involve enzyme catalyzed reactions. Thus, in one example, glucose may be degraded enzymatically.

Advantageously, the compositions described herein may promote the degradation (e.g. break down) of glucose in the gut, reducing absorption of glucose through the intestinal tract. A similar effect and corresponding biochemical findings are seen when drugs belonging to the class of SGLT-1 inhibitors (α-glucosidase inhibitors, e.g. acarbose or voglibiose) are used in routine diabetes treatment. SGLT-1 inhibitors actively withhold the glucose molecules in the intestine tract, where they finally get metabolized by bacteria populating the large bowel, with methane gas as the final end product. Advantageously, it is suggested that AB001 has four glucose-controlling actions as discussed above. It is suggested that supplementation with AB001 means that less glucose reaches the blood.

“Metabolism”, as used herein, is a term used to describe all biochemical reactions involved in maintaining the living state of cells and organisms. For example, metabolism includes all the biochemical reactions involved in converting one molecule into another (to essentially maintain the living state of a cell or an organism). Metabolism includes processes for cell growth, reproduction, response to the environment, survival mechanisms, sustenance, and maintenance of cell structure and integrity. The biochemical reactions involved in metabolism utilize various enzymes.

In one example, glucose may be metabolized (i.e. glucose may be degraded via metabolism). Accordingly, in one example, a glucose metabolizing composition comprising one or more bacterial species of the Bacillus genus, rice bran, L-cysteine and a high molecular weight low osmolality carbohydrate (e.g. dextrin) for use according to the present invention (e.g. for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) is provided. As is known to the skilled person, metabolism may be enzymatic thus, in some examples, glucose may be metabolized enzymatically.

Metabolism may be categorized into two: catabolism and anabolism. Catabolism includes a series of degradative biochemical reactions that break down complex molecules into smaller units, usually releasing energy in the process. For example, catabolism may be used to refer to all biochemical or enzymatic reactions involved in the breakdown of organic or inorganic materials such as proteins, sugars, fatty acids, etc. Anabolism includes a sequence of biochemical reactions that constructs or synthesizes molecules from smaller units, usually requiring the input of energy (ATP) in the process. Catabolism thus refers to destructive biochemical reactions which occur in an organism whereas metabolism refers to the whole set of biochemical reactions in the organism, which can be either constructive or destructive.

In some examples, the degradation of glucose via metabolism may be considered as catabolism. Accordingly, in some examples, glucose may be catabolized (i.e glucose may be degraded via catabolism). In a further example, glucose may be catabolized enzymatically.

Typically, in the context of the present invention, glucose is degraded in a subject. By way of non-limiting example, glucose may be broken down enzymatically, metabolized or catabolized in a subject. Where glucose degradation occurs in a subject (e.g. particularly in the duodenum and/or small intestine) this may be referred to digestion. In some examples, glucose may be degraded by biochemical digestion.

Accordingly, in one example, a glucose degrading composition for use according to the present invention comprising one or more bacterial species of the Bacillus genus, rice bran, L-cysteine and a high molecular weight low osmolality carbohydrate (e.g. dextrin) is provided herein.

In another example, a glucose metabolizing composition for use according to the present invention comprising one or more bacterial species of the Bacillus genus, rice bran, L-cysteine and a high molecular weight low osmolality carbohydrate (e.g. dextrin) is provided herein.

As discussed elsewhere herein, the compositions for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) comprise one or more bacterial species of the Bacillus genus. For example, the composition for use according to the present invention may comprise two or more bacterial species of the Bacillus genus. In another example, the composition for use according to the present invention may comprise three or more bacterial species of the Bacillus genus. In a further example, the composition for use according to the present invention may comprise four or more bacterial species of the Bacillus genus. In another example, the composition for use according to the present invention may comprise five or more bacterial species of the Bacillus genus. In a further example, the composition for use according to the present invention may comprise six or more bacterial species of the Bacillus genus. In another example, the composition for use according to the present invention may comprise seven or more bacterial species of the Bacillus genus.

As is known in the art, a genus is made up of several species. The genus “Bacillus” thus includes all species within the genus “Bacillus,” known to those of skill in the art, including but not limited to Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus velezensis, Bacillus sp MT 03, Bacillus atrophaeus, and Bacillus thuringiensis. It is recognized that the genus Bacillus continues to undergo taxonomical reorganization. Thus, it is intended that the genus includes species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

Species within the genus Bacillus are gram-positive bacteria classified as members of the Family Bacillaceae, Order Bacillales, Class Bacilli. As used herein, “Bacillus species” (“Bacillus sp.”) refers to a species within the genus “Bacillus”.

Bacillus species found to be particularly important in the context of the present invention include Bacillus subtilis (B. subtilis) and Bacillus coagulans (B. coagulans). As described in the examples below, these bacteria are abundant in the composition for use according to the present invention and are particularly effective at degrading (e.g. metabolizing) glucose especially within the gut.

Accordingly, in one example, the one or more bacterial species of the Bacillus genus is selected from B. subtilis and B. coagulans.

In another example, the composition for use according to the present invention comprises B. subtilis and B. coagulans.

Any suitable B. subtilis strains and/or B. coagulans strains may be used in accordance with the present invention. A person skilled in the art would readily be able to identify suitable strains. Bacillus subtilis strain DFM 0326 (LMG P-32899), Bacillus subtilis strain DFM 1015 (LMG P-32900) and Bacillus coagulans strain DFM 0705 (LMG P-32921) have been found to be particularly important in the context of the present invention.

A Bacillus subtilis strain deposited under LMG-P accession number 32899 may be referred to herein as “DFM 0326” or “strain DFM 0326”. Bacillus subtilis strain DFM 0326 (deposited under LMG-P accession number 32899) was deposited at the Belgian Coordinated Collections of Micro-organisms (BCCM), Laboratorium voor Microbiologie—Bacteriënverzameling (LMG), Universiteit Gent, K. L. Ledeganckstraat 35, 9000 Gent, Belgium under The Budapest Treaty of 1977 on 22 Nov. 2022.

A Bacillus subtilis strain deposited under LMG-P accession number 32900 may be referred to herein as “DFM 1015” or “strain DFM 1015”. Bacillus subtilis strain DFM 1015 (deposited under LMG-P accession number 32900) was deposited at the Belgian Coordinated Collections of Micro-organisms (BCCM), Laboratorium voor Microbiologie—Bacteriênverzameling (LMG), Universiteit Gent, K. L. Ledeganckstraat 35, 9000 Gent, Belgium under The Budapest Treaty of 1977 on 22 Nov. 2022.

A Bacillus coagulans strain deposited under LMG-P accession number 32921 may be referred to herein as “DFM 0705” or “strain DFM 0705”. Bacillus coagulans strain DFM 0705 (deposited under LMG-P accession number 32921) was deposited at the Belgian Coordinated Collections of Micro-organisms (BCCM), Laboratorium voor Microbiologie—Bacteriënverzameling (LMG), Universiteit Gent, K. L. Ledeganckstraat 35, 9000 Gent, Belgium under The Budapest Treaty of 1977 on 14 Dec. 2022.

Accordingly, in some examples the B. subtilis species may be selected from the group consisting of: Bacillus subtilis strain DFM 0326 (LMG P-32899) and Bacillus subtilis strain DFM 1015 (LMG P-32900); and/or the B. coagulans species may be Bacillus coagulans strain DFM 0705 (LMG P-32921).

Accordingly, in examples where the composition comprises B. subtilis and B. coagulans, the B. subtilis species may be Bacillus subtilis strain DFM 0326 (LMG P-32899) and the B. coagulans species may be Bacillus coagulans strain DFM 0705 (LMG P-32921).

In another example where the composition comprises B. subtilis and B. coagulans, the B. subtilis species may be Bacillus subtilis strain DFM 1015 (LMG P-32900) and the B. coagulans species may be Bacillus coagulans strain DFM 0705 (LMG P-32921).

In some examples, the composition according to the invention may comprise the B. subtilis strain DFM 0326 (LMG P-32899) and Bacillus subtilis strain DFM 1015 (LMG P-32900) in combination.

In another example where the composition comprises B. subtilis and B. coagulans, the composition may comprise the B. subtilis strain DFM 0326 (LMG P-32899) and Bacillus subtilis strain DFM 1015 (LMG P-32900) and the B. coagulans species strain DFM 0705 (LMG P-32921). Notably, the compositions used in the examples provided herein comprise the B. subtilis strain DFM 0326 (LMG P-32899) and Bacillus subtilis strain DFM 1015 (LMG P-32900) and the B. coagulans strain DFM 0705 (LMG P-32921).

Accordingly, the composition for use according to the present invention may comprise Bacillus subtilis strain DFM 0326 (LMG P-32899), Bacillus subtilis strain DFM 1015 (LMG P-32900) and Bacillus coagulans strain DFM 0705 (LMG P-32921).

The inventors also identified several other bacterial species that are particularly relevant in the context of the invention. Accordingly, in one example, the composition for use according to the present invention may further comprise one or more bacterial species selected from the group consisting of: Bacillus amyloliquefaciens, Bacillus velezensis, Bacillus sp MT 03, Bacillus atrophaeus, and Pediococcus pentosaceus. In one example, the composition for use according to the present invention may further comprise one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa). In another example, the composition for use according to the present invention may further comprise two or more, or three or more, or four or more bacterial species selected from the group consisting of: Bacillus amyloliquefaciens, Bacillus velezensis, Bacillus sp MT 03, Bacillus atrophaeus, and Pediococcus pentosaceus. In another example, the composition for use according to the present invention may further comprise two or more, or three or more, or four or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa).

In another example, the composition for use according to the present invention may further comprise Bacillus amyloliquefaciens, Bacillus velezensis, Bacillus sp MT 03, Bacillus atrophaeus, and Pediococcus pentosaceus. In another example, the composition for use according to the present invention may further comprise Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa).

The bacterial strains included in the composition described herein were identified to preferably and effectively metabolize glucose. Advantageously, it is suggested that a result of AB001 supplementation is a lower absorption of glucose through the intestine tract. In consequence, it is suggested that supplementation with AB001 means that less glucose reaches the blood.

The composition for use according to the present invention comprises an appropriate amount or concentration of the one or more bacterial species of the Bacillus genus. The amount or concentration of the one or more bacterial species of the Bacillus genus may therefore be described by reference to the number of colony forming units (cfu) per gram of composition (cfu/g) or by the total number of colony forming units (cfu) per dose of composition (in other words the cfu per effective dose). As would be clear to a person of skill in the art, and as described in more detail elsewhere herein, a dose may include one or more dosage units (e.g. 2 dosage units). In examples wherein a plurality of dosage units are used to provide an effective dose, the cfu/dose corresponds to the total cfu over the plurality of dosage units.

In one example, the composition described herein comprises about 10,000 cfu/g of bacteria of the Bacillus genus to about 1×10⁸ cfu/g of bacteria of the Bacillus genus.

In one example, the composition described herein comprises at least about 1×10⁸ cfu/g of bacteria of the Bacillus genus. In another example, the composition described herein comprises at least about 1×10⁷ cfu/g of bacteria of the Bacillus genus. In another example, the composition described herein comprises at least about 1×10⁶ cfu/g of bacteria of the Bacillus genus. In a further example, the composition described herein comprises at least about 1×10⁵ cfu/g of bacteria of the Bacillus genus.

In one example, the composition described herein comprises at least about 10,000 cfu/g (i.e. at least about 1.0×10⁴ cfu/g) of bacteria of the Bacillus genus.

In another example, the composition described herein comprises at least about 11,000 cfu/g (i.e. at least about 1.1×10⁴ cfu/g) of bacteria of the Bacillus genus. In a further example, the composition described herein comprises at least about 12,000 cfu/g (i.e. at least about 1.2×10⁴ cfu/g) of bacteria of the Bacillus genus.

In another example, the composition described herein comprises at least about 13,000 cfu/g (i.e. at least about 1.3×10⁴ cfu/g) of bacteria of the Bacillus genus.

In another example, the composition described herein comprises at least about 14,000 cfu/g (i.e. at least about 1.4×10⁴ cfu/g) of bacteria of the Bacillus genus. In another example, the composition described herein comprises at least about 15,000 cfu/g (i.e. at least about 1.5×10⁴ cfu/g) of bacteria of the Bacillus genus.

In one example, the composition described herein comprises about 1×10⁸ cfu/g of bacteria of the Bacillus genus. In another example, the composition described herein comprises about 1×10⁷ cfu/g of bacteria of the Bacillus genus. In another example, the composition described herein comprises about 1×10⁶ cfu/g of bacteria of the Bacillus genus.

In a further example, the composition described herein comprises about 1×10⁵ cfu/g of bacteria of the Bacillus genus.

In one example, the composition described herein comprises about 10,000 cfu/g (i.e. about 1.0×10⁴ cfu/g) of bacteria of the Bacillus genus.

In another example, the composition described herein comprises about 11,000 cfu/g (i.e. about 1.1×10⁴ cfu/g) of bacteria of the Bacillus genus. In a further example, the composition described herein comprises about 12,000 cfu/g (i.e. about 1.2×10⁴ cfu/g) of bacteria of the Bacillus genus.

In another example, the composition described herein comprises about 13,000 cfu/g (i.e. about 1.3×10⁴ cfu/g) of bacteria of the Bacillus genus.

In another example, the composition described herein comprises about 14,000 cfu/g (i.e. about 1.4×10⁴ cfu/g) of bacteria of the Bacillus genus. In a further example, the composition described herein comprises about 15,000 cfu/g (i.e. about 1.5×10⁴ cfu/g) of bacteria of the Bacillus genus.

A person of skill in the art would readily be able to determine the amount or concentration of bacteria present in a composition using routine methods known in the art. For instance, the total viable count (TVC) of Bacillus live cells for example can be determined by established cultivation methods based on specific Bacillus media, such as Chrome Select agar. Alternative methods are known in the art.

In one example, the composition described herein comprises about 5,000 cfu of bacteria of the Bacillus genus/dose to about 1×10⁸ cfu of bacteria of the Bacillus genus/dose.

In one example, the composition described herein comprises at least about 1×10⁸ cfu of bacteria of the Bacillus genus/dose. In another example, the composition described herein comprises at least about 1×10⁷ cfu of bacteria of the Bacillus genus/dose. In another example, the composition described herein comprises at least about 1×10⁶cfu of bacteria of the Bacillus genus/dose.

In a further example, the composition described herein comprises at least about 1×10⁵ cfu of bacteria of the Bacillus genus/dose.

In one example, the composition described herein comprises at least about 5,000 cfu (i.e. at least about 0.5×10⁴ cfu) of bacteria of the Bacillus genus/dose.

In another example, the composition described herein comprises at least about 10,000 cfu (i.e. at least about 1.0×10⁴cfu) of bacteria of the Bacillus genus/dose. In another example, the composition described herein comprises at least about 11,000 cfu (i.e. at least about 1.1×10⁴ cfu) of bacteria of the Bacillus genus/dose. In a further example, the composition described herein comprises at least about 12,000 cfu (i.e. at least about 1.2×10⁴ cfu) of bacteria of the Bacillus genus/dose. In another example, the composition described herein comprises at least about 13,000 cfu (i.e. at least about 1.3×10⁴ cfu) of bacteria of the Bacillus genus/dose. In another example, the composition described herein comprises at least about 14,000 cfu (i.e. at least about 1.4×10⁴ cfu) of bacteria of the Bacillus genus/dose. In another example, the composition described herein comprises at least about 15,000 cfu (i.e. at least about 1.5×10⁴ cfu) of bacteria of the Bacillus genus/dose.

In one example, the composition described herein comprises about 1×10⁸ cfu of bacteria of the Bacillus genus/dose. In another example, the composition described herein comprises about 1×10⁷ cfu of bacteria of the Bacillus genus/dose. In another example, the composition described herein comprises about 1×10⁶ cfu of bacteria of the Bacillus genus/dose.

In a further example, the composition described herein comprises about 1×10⁵ cfu of bacteria of the Bacillus genus/dose.

In one example, the composition described herein comprises about 5,000 cfu (i.e. about 0.5×10⁴ cfu) of bacteria of the Bacillus genus/dose. In one example, the composition described herein comprises about 10,000 cfu (i.e. about 1.0×10⁴ cfu) of bacteria of the Bacillus genus/dose. In another example, the composition described herein comprises about 11,000 cfu (i.e. about 1.1×10⁴ cfu) of bacteria of the Bacillus genus/dose. In a further example, the composition described herein comprises about 12,000 cfu (i.e. about 1.2×10⁴ cfu) of bacteria of the Bacillus genus/dose. In another example, the composition described herein comprises about 13,000 cfu (i.e. about 1.3×10⁴ cfu) of bacteria of the Bacillus genus/dose. In another example, the composition described herein comprises about 14,000 cfu (i.e. about 1.4×10⁴ cfu) of bacteria of the Bacillus genus/dose. In a further example, the composition described herein comprises about 15,000 cfu (i.e. about 1.5×10⁴ cfu) of bacteria of the Bacillus genus/dose.

As would be clear to the skilled person, the amount or concentration of the one or more bacterial species of the Bacillus genus in the composition may be made up from any individual species of the Bacillus genus or any combination of species of the Bacillus genus. For example, the amount or concentration of the one or more bacterial species of the Bacillus genus in the composition may be made up from entirely one Bacillus species (e.g. the concentration may be made up from entirely B. subtilis or entirely B. coagulans). Alternatively, the amount or concentration of the one or more bacterial species of the Bacillus genus in the composition may be made up from two or more, three or more, four or more, or five or more bacterial species of the Bacillus genus (e.g. the concentration may be made up from B. subtilis and B. coagulans). Accordingly, in one example, the amount or concentration of the one or more bacterial species of the Bacillus genus in the composition refers to the amount or concentration of the combination of the Bacillus species present.

In some examples, the composition provided herein comprises an appropriate concentration of bacteria, wherein a proportion of the concentration is made up from one or more bacterial species of the Bacillus genus (e.g. B. subtilis and/or B. coagulans). In one example, the composition described herein comprises about 10,000 cfu/g (i.e. about 1.0×10⁴ cfu/g) of bacteria to about 1×10⁸cfu/g of bacteria. In one example, the composition described herein comprises at least about 1×10⁸cfu/g of bacteria. In another example, the composition described herein comprises at least about 1×10⁷ cfu/g of bacteria. In another example, the composition described herein comprises at least about 1×10⁶ cfu/g of bacteria. In a further example, the composition described herein comprises at least about 1×10⁵ cfu/g of bacteria. In one example, the composition described herein comprises at least about 10,000 cfu/g (i.e. at least about 1.0×10⁴ cfu/g) of bacteria. In another example, the composition described herein comprises at least about 11,000 cfu/g (i.e. at least about 1.1×10⁴ cfu/g) of bacteria. In a further example, the composition described herein comprises at least about 12,000 cfu/g (i.e. at least about 1.2×10⁴cfu/g) of bacteria. In another example, the composition described herein comprises at least about 13,000 cfu/g (i.e. at least about 1.3×10⁴ cfu/g) of bacteria. In another example, the composition described herein comprises at least about 14,000 cfu/g (i.e. at least about 1.4×10⁴ cfu/g) of bacteria. In another example, the composition described herein comprises at least about 15,000 cfu/g (i.e. at least about 1.5×10⁴ cfu/g) of bacteria. In one example, the composition described herein comprises about 1×10⁸ cfu/g of bacteria. In another example, the composition described herein comprises about 1×10⁷ cfu/g of bacteria. In another example, the composition described herein comprises about 1×10⁶ cfu/g of bacteria. In a further example, the composition described herein comprises about 1×10⁵ cfu/g of bacteria. In one example, the composition described herein comprises about 10,000 cfu/g (i.e. about 1.0×10⁴ cfu/g) of bacteria. In another example, the composition described herein comprises about 11,000 cfu/g (i.e. about 1.1×10⁴ cfu/g) of bacteria. In a further example, the composition described herein comprises about 12,000 cfu/g (i.e. about 1.2×10⁴ cfu/g) of bacteria. In another example, the composition described herein comprises about 13,000 cfu/g (i.e. about 1.3×10⁴ cfu/g) of bacteria. In another example, the composition described herein comprises about 14,000 cfu/g (i.e. about 1.4×10⁴ cfu/g) of bacteria. In a further example, the composition described herein comprises about 15,000 cfu/g (i.e. about 1.5×10⁴ cfu/g) of bacteria.

In some examples, the composition described herein comprises an appropriate amount of bacteria, wherein a proportion of the amount is made up from one or more bacterial species of the Bacillus genus (e.g. B. subtilis and/or B. coagulans). In one example, the composition described herein comprises about 5,000 cfu of bacteria/dose to about 1×10⁸ cfu of bacteria/dose. In one example, the composition described herein comprises at least about 1×10⁸ cfu of bacteria/dose. In another example, the composition described herein comprises at least about 1×10⁷ cfu of bacteria/dose. In another example, the composition described herein comprises at least about 1×10⁶ cfu of bacteria/dose. In a further example, the composition described herein comprises at least about 1×10⁵ cfu of bacteria/dose. In one example, the composition described herein comprises at least about 5,000 cfu (i.e. at least about 0.5×10⁴ cfu) of bacteria/dose. In another example, the composition described herein comprises at least about 10,000 cfu (i.e. at least about 1.0×10⁴ cfu) of bacteria/dose. In another example, the composition described herein comprises at least about 11,000 cfu (i.e. at least about 1.1×10⁴ cfu) of bacteria/dose. In a further example, the composition described herein comprises at least about 12,000 cfu (i.e. at least about 1.2×10⁴ cfu) of bacteria/dose. In another example, the composition described herein comprises at least about 13,000 cfu (i.e. at least about 1.3×10⁴ cfu) of bacteria/dose. In another example, the composition described herein comprises at least about 14,000 cfu (i.e. at least about 1.4×10⁴ cfu) of bacteria/dose. In another example, the composition described herein comprises at least about 15,000 cfu (i.e. at least about 1.5×10⁴ cfu) of bacteria/dose. In one example, the composition described herein comprises about 1×10⁸ cfu of bacteria/dose. In another example, the composition described herein comprises about 1×10⁷ cfu of bacteria/dose. In another example, the composition described herein comprises about 1×10⁶ cfu of bacteria/dose. In a further example, the composition described herein comprises about 1×10⁵ cfu of bacteria/dose. In one example, the composition described herein comprises about 5,000 cfu (i.e. about 0.5×10⁴ cfu) of bacteria/dose. In one example, the composition described herein comprises about 10,000 cfu (i.e. about 1.0×10⁴ cfu) of bacteria/dose. In another example, the composition described herein comprises about 11,000 cfu (i.e. about 1.1×10⁴ cfu) of bacteria/dose. In a further example, the composition described herein comprises about 12,000 cfu (i.e. about 1.2×10⁴ cfu) of bacteria/dose. In another example, the composition described herein comprises about 13,000 cfu (i.e. about 1.3×10⁴ cfu) of bacteria/dose. In another example, the composition described herein comprises about 14,000 cfu (i.e. about 1.4×10⁴ cfu) of bacteria/dose. In a further example, the composition described herein comprises about 15,000 cfu (i.e. about 1.5×10⁴ cfu) of bacteria/dose.

As would be clear to the skilled person, the amount or concentration of bacteria (wherein a proportion is made up from one or more bacterial species of the Bacillus genus (e.g. B. subtilis and/or B. coagulans) may further comprise any appropriate individual species or any combination of species. For example, the amount or concentration of bacteria (wherein a proportion is made up from one or more bacterial species of the Bacillus genus (e.g. B. subtilis and/or B. coagulans)) may further comprise a proportion of one or more bacterial species selected from the group consisting of: Bacillus amyloliquefaciens, Bacillus velezensis, Bacillus sp MT 03, Bacillus atrophaeus, and Pediococcus pentosaceus. For example, the amount or concentration of bacteria (wherein a proportion is made up from one or more bacterial species of the Bacillus genus (e.g. B. subtilis and/or B. coagulans)) may further comprise a proportion of one or more bacterial species selected from the group consisting of: Bacillus amyloliquefaciens, Bacillus velezensis, Bacillus sp MT 03, Bacillus atrophaeus, Pediococcus pentosaceus, and genera from Saccharomycetes, (e.g. Brettanomyces bruxellensis, and/or Millerozoma farinosa).

In one example, the one or more bacterial species of the Bacillus genus is genetically modified.

As used herein, “genetic modification” and “genetic engineering” refer to the direct manipulation (e.g. modification) of one or more genes, for example, using recombinant DNA technology. Traditionally, humans have manipulated genomes indirectly by controlling breeding and selecting offspring with desired traits however genetic engineering involves direct manipulation (e.g. modification) of one or more genes. For example, a gene from another species may be added to an organism's genome to give it a desired phenotype.

In other examples, the one or more bacterial species of the Bacillus genus is not genetically modified. In other words, the one or more bacterial species of the Bacillus genus may be naturally occurring.

The one or more bacterial species of the Bacillus genus may be present within a microbial consortium. “Microbial consortium”, as used herein, refers to a group of microbes (e.g. bacteria) wherein the group comprises two or more distinct microbes (e.g. two of more bacteria which may be from the same species (e.g. two or more distinct strains) or distinct species (e.g. two or more distinct species).

The microbial consortium may be a naturally occurring microbial consortium (e.g. a consortium that is naturally produced during fermentation of a cereal, such as rice bran). In other words, the one or more bacterial species of the Bacillus genus may be part of the composition by virtue of the presence of a fermented cereal, such as rice bran (with its associated naturally produced microbial consortium) in the composition. The one or more bacterial species of the Bacillus genus may therefore be a natural component of a fermented cereal, such as rice bran.

The inventors have found that Bacillus subtilis strain DFM 0326 (LMG P-32899), Bacillus subtilis strain DFM 1015 (LMG P-32900) and Bacillus coagulans strain DFM 0705 (LMG P-32921) can be isolated from the compositions (in particular, the fermented rice bran) used in the examples provided herein. Accordingly, the B. subtilis strain DFM 0326 (LMG P-32899) and Bacillus subtilis strain DFM 1015 (LMG P-32900), and the B. coagulans strain DFM 0705 (LMG P-32921) may be part of the composition described herein by virtue of the presence of a fermented cereal, such as rice bran (with its associated naturally produced microbial consortium), in the composition.

In one example, the one or more bacterial species of the Bacillus genus selected from B. subtilis and B. coagulans may be (part of) a naturally occurring microbial consortium (e.g. a consortium that is naturally produced during fermentation of a cereal, such as rice bran). In other words, the one or more bacterial species of the Bacillus genus selected from B. subtilis and B. coagulans may be part of the composition by virtue of the presence of a fermented cereal, such as rice bran (with its associated naturally produced microbial consortium) in the composition. The one or more bacterial species of the Bacillus genus selected from B. subtilis and B. coagulans may therefore be a natural component of a fermented cereal, such as rice bran.

In another example, the one or more bacterial species selected from the group consisting of: Bacillus amyloliquefaciens, Bacillus velezensis, Bacillus sp MT 03, Bacillus atrophaeus, and Pediococcus pentosaceus may be (part of) a naturally occurring microbial consortium (e.g. a consortium that is naturally produced during fermentation of a cereal, such as rice bran). In other words, the one or more bacterial species selected from the group consisting of: Bacillus amyloliquefaciens, Bacillus velezensis, Bacillus sp MT 03, Bacillus atrophaeus, and Pediococcus pentosaceus may be part of the composition by virtue of the presence of a fermented cereal, such as rice bran (with its associated naturally produced microbial consortium) in the composition. The one or more bacterial species selected from the group consisting of: Bacillus amyloliquefaciens, Bacillus velezensis, Bacillus sp MT 03, Bacillus atrophaeus, and Pediococcus pentosaceus may therefore be a natural component of a fermented cereal, such as rice bran.

In another example, the one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g.

Brettanomyces bruxellensis, and/or Millerozoma farinose) may be (part of) a naturally occurring microbial consortium (e.g. a consortium that is naturally produced during fermentation of a cereal, such as rice bran). In other words, the one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa may be part of the composition by virtue of the presence of a fermented cereal, such as rice bran (with its associated naturally produced microbial consortium) in the composition. The one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa) may therefore be a natural component of a fermented cereal, such as rice bran.

In another example, the one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may be (part of) a naturally occurring microbial consortium (e.g. a consortium that is naturally produced during fermentation of a cereal, such as rice bran). In other words, the one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may be part of the composition by virtue of the presence of a fermented cereal, such as rice bran (with its associated naturally produced microbial consortium) in the composition. The one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may therefore be a natural component of a fermented cereal, such as rice bran.

In another example, the one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa) may be (part of) a naturally occurring microbial consortium (e.g. a consortium that is naturally produced during fermentation of a cereal, such as rice bran). In other words, the one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinosa) may be part of the composition by virtue of the presence of a fermented cereal, such as rice bran (with its associated naturally produced microbial consortium) in the composition. The one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, Pediococcus pentosaceus and genera from Saccharomycetes, (e.g. Brettanomyces bruxellensis, and/or Millerozoma farinose) may therefore be a natural component of a fermented cereal, such as rice bran.

As would be clear to a person of skill in the art, the composition for use according to the invention is not limited to naturally occurring microbial consortia. Accordingly, the microbial consortia discussed above may also be generated artificially, for example by combining one or more bacterial isolates together. In one example, the one or more bacterial species of the Bacillus genus (e.g. B. subtilis and/or B. coagulans) may therefore be added to the composition individually.

The composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) may therefore also include a cereal such as rice bran (e.g. fermented rice bran) as discussed in more detail elsewhere herein. The fermented rice bran, for example, may be the natural source of the one or more bacterial species of the Bacillus genus (e.g. B. subtilis and/or B. coagulans) present within the composition.

As described elsewhere herein, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) comprises L-cysteine in combination with the one or more bacterial species of the Bacillus genus (e.g. wherein the bacteria are added to the composition as a bacterial supplement or as a natural product e.g. as part of fermented rice bran).

L-cysteine (L-cys) is a non-essential amino acid and thus is one of the building blocks required for the synthesis of proteins. It contains sulphur in the form of a thiol group (—SH) at the end of its side chain. The —SH group is responsible for the high reactive capacity of the amino acid, and therefore is responsible for many of its biological functions in human beings. L-cysteine occupies a key position in sulfur metabolism in all organisms and is used in the synthesis of proteins, glutathione, biotin, lipoic acid, methionine and other sulfur-containing metabolites. In addition, L-cysteine serves as a precursor for the biosynthesis of coenzyme A. The biosynthesis of L-cysteine has been studied in detail in bacteria, especially in enterobacteria. The amino acid L-cysteine is not only of biological importance but is also of economic importance. It is used, for example, as a food additive (in particular in the baking industry), as a starting material in cosmetics, and as a starting material for the preparation of active pharmaceutical ingredients (in particular N-acetyl-cysteine and S-carboxymethyl-cysteine).

As would be clear to a person of skill in the art, reference to L-cysteine herein refers to the amino acid L-cysteine in any suitable form. The term “L-cysteine” therefore encompasses L-cysteine in free form as well as L-cysteine salts.

In the context of the present invention, in one example L-cysteine can be in the free form, a salt thereof, or a mixture thereof. Examples of the salt include, for example, sulfate, hydrochloride, carbonate, ammonium salt, sodium salt, and potassium salt. In one example, the L-cysteine is a crystal fraction of >0.1 mm.

L-cysteine is available from several suppliers and can be readily sourced by the skilled person. Furthermore, the skilled person would readily be able to detect the presence of L-cysteine in a substance (e.g. in a composition described herein) using methods known in the art. For instance, L-Cysteine crystals may be macroscopically detected in the composition described herein as white particles. HPLC, High-Performance Liquid Chromatography, on an Inertsil ODs-3 column is an established method for detection of L-cysteine.

L-cysteine may be obtained industrially by hydrolysis of animal materials, such as poultry feathers or hog hair. In contrast, synthetic L-cys may be obtained from fermentation of genetically modified E. coli or Pseudomonas thiazolinophilum. Accordingly, in some examples, L-cysteine is of animal origin. In other examples, L-cysteine is of synthetic origin.

In one example, L-cysteine is of vegetable origin.

In some examples, the composition described herein may comprise an L-cysteine derivative instead of or in addition to L-cysteine.

L-cysteine derivatives are well known to persons of skill in the art. N-acetyl cysteine (NAC) is an N-acetylated form of the amino acid L-cysteine thus is an example of an L-cysteine derivative. Accordingly, in some examples the composition described herein may comprise N-acetylcysteine (NAC). NAC is readily available in the art.

L-cysteine is present in the composition described herein at an appropriate concentration or amount. The amount or concentration of L-cysteine may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of L-cysteine over the plurality of dosage units.

In one example, the composition comprises about 10% w/w of L-cysteine to about 40% w/w of L-cysteine.

In another example, the composition comprises about 15% w/w of L-cysteine to about 35% w/w of L-cysteine. In another example, the composition comprises about 20% w/w of L-cysteine to about 30% w/w of L-cysteine.

In one example, the composition comprises at least about 10% w/w of L-cysteine.

In another example, the composition comprises at least about 15% w/w of L-cysteine.

In a further example, the composition comprises at least about 20% w/w of L-cysteine.

In another example, the composition comprises at least about 25% w/w of L-cysteine.

In another example, the composition comprises at least about 30% w/w of L-cysteine. In a further example, the composition comprises at least about 35% w/w of L-cysteine. In a further example, the composition comprises at least about 40% w/w of L-cysteine.

In some examples, the composition comprises about 10% w/w of L-cysteine.

In another example, the composition comprises about 15% w/w of L-cysteine.

In another example, the composition comprises about 20% w/w of L-cysteine.

In a further example, the composition comprises about 25% w/w of L-cysteine. In another example, the composition comprises about 30% w/w of L-cysteine. In another example, the composition comprises about 35% w/w of L-cysteine. In a further example, the composition comprises about 40% w/w of L-cysteine.

In one example, the composition comprises about 38 mg of L-cysteine/dose to about 200 mg of L-cysteine/dose.

In one example, the composition comprises at least about 38 mg of L-cysteine/dose.

In one example, the composition comprises at least about 50 mg of L-cysteine/dose.

In one example, the composition comprises at least about 75 mg of L-cysteine/dose. In one example, the composition comprises at least about 100 mg of L-cysteine/dose.

In one example, the composition comprises at least about 150 mg of L-cysteine/dose.

In one example, the composition comprises at least about 160 mg of L-cysteine/dose.

In one example, the composition comprises at least about 180 mg of L-cysteine/dose.

In one example, the composition comprises about 38 mg of L-cysteine/dose.

In one example, the composition comprises about 50 mg of L-cysteine/dose. In one example, the composition comprises about 75 mg of L-cysteine/dose. In one example, the composition comprises about 100 mg of L-cysteine/dose.

In one example, the composition comprises about 150 mg of L-cysteine/dose.

In one example, the composition comprises about 160 mg of L-cysteine/dose. In one example, the composition comprises about 180 mg of L-cysteine/dose.

As described elsewhere herein, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) comprises L-cysteine in combination with the one or more bacterial species of the Bacillus genus and a high molecular weight low osmolality carbohydrate (e.g. dextrin).

As used herein, a “high molecular weight low osmolality carbohydrate” refers to a carbohydrate with a molecular weight of about 500,000 g/mol to about 700,000 g/mol wherein the osmolality of the carbohydrate is low. A person of skill in the art would readily be able to identify appropriate carbohydrates with a low osmolality using routine tests known in the art. For the avoidance of doubt, as used herein, a low osmolality carbohydrate is a carbohydrate that has about 50% greater glycogen recovery than maltodextrin.

The high molecular weight low osmolality carbohydrate is present in the composition described herein at an appropriate concentration or amount. The amount or concentration of the high molecular weight low osmolality carbohydrate may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of the high molecular weight low osmolality carbohydrate over the plurality of dosage units.

In one example, the composition comprises about 0.5% w/w to about 5% w/w of high molecular weight low osmolality carbohydrate.

In another example, the composition comprises about 0.5% w/w to about 3% w/w of high molecular weight low osmolality carbohydrate. In one example, the composition comprises about 0.5% w/w to about 2% w/w of high molecular weight low osmolality carbohydrate.

In one example, the composition comprises at least about 0.5% w/w of high molecular weight low osmolality carbohydrate.

In another example, the composition comprises at least about 1% w/w of high molecular weight low osmolality carbohydrate. In another example, the composition comprises at least about 1.5% w/w of high molecular weight low osmolality carbohydrate. In a further example, the composition comprises at least about 2% w/w of high molecular weight low osmolality carbohydrate. In another example, the composition comprises at least about 2.5% w/w of high molecular weight low osmolality carbohydrate. In another example, the composition comprises at least about 3% w/w of high molecular weight low osmolality carbohydrate. In another example, the composition comprises at least about 3.5% w/w of high molecular weight low osmolality carbohydrate. In a further example, the composition comprises at least about 4% w/w of high molecular weight low osmolality carbohydrate. In another example, the composition comprises at least about 4.5% w/w of high molecular weight low osmolality carbohydrate. In a further example, the composition comprises at least about 5% w/w of high molecular weight low osmolality carbohydrate.

In one example, the composition comprises about 0.5% w/w of high molecular weight low osmolality carbohydrate.

In another example, the composition comprises about 1% w/w of high molecular weight low osmolality carbohydrate. In another example, the composition comprises about 1.5% w/w of high molecular weight low osmolality carbohydrate. In a further example, the composition comprises about 2% w/w of high molecular weight low osmolality carbohydrate. In another example, the composition comprises about 2.5% w/w of high molecular weight low osmolality carbohydrate. In another example, the composition comprises about 3% w/w of high molecular weight low osmolality carbohydrate. In another example, the composition comprises about 3.5% w/w of high molecular weight low osmolality carbohydrate. In a further example, the composition comprises about 4% w/w of high molecular weight low osmolality carbohydrate. In another example, the composition comprises about 4.5% w/w of high molecular weight low osmolality carbohydrate. In a further example, the composition comprises about 5% w/w of high molecular weight low osmolality carbohydrate.

In one example, the composition comprises about 2 mg to about 50 mg of high molecular weight low osmolality carbohydrate/dose.

In one example, the composition comprises at least about 2 mg of high molecular weight low osmolality carbohydrate/dose.

In one example, the composition comprises at least about 4 mg of high molecular weight low osmolality carbohydrate/dose.

In one example, the composition comprises at least about 10 mg of high molecular weight low osmolality carbohydrate/dose. In one example, the composition comprises at least about 15 mg of high molecular weight low osmolality carbohydrate/dose. In one example, the composition comprises at least about 20 mg of high molecular weight low osmolality carbohydrate/dose. In one example, the composition comprises at least about 25 mg of high molecular weight low osmolality carbohydrate/dose. In one example, the composition comprises at least about 30 mg of high molecular weight low osmolality carbohydrate/dose. In one example, the composition comprises at least about 35 mg of high molecular weight low osmolality carbohydrate/dose. In one example, the composition comprises at least about 40 mg of high molecular weight low osmolality carbohydrate/dose. In one example, the composition comprises at least about 45 mg of high molecular weight low osmolality carbohydrate/dose.

In one example, the composition comprises about 2 mg of high molecular weight low osmolality carbohydrate/dose.

In one example, the composition comprises about 4 mg of high molecular weight low osmolality carbohydrate/dose.

In one example, the composition comprises about 10 mg of high molecular weight low osmolality carbohydrate/dose. In one example, the composition comprises about 15 mg of high molecular weight low osmolality carbohydrate/dose. In one example, the composition comprises about 20 mg of high molecular weight low osmolality carbohydrate/dose. In one example, the composition comprises about 25 mg of high molecular weight low osmolality carbohydrate/dose. In one example, the composition comprises about 30 mg of high molecular weight low osmolality carbohydrate/dose. In one example, the composition comprises about 35 mg of high molecular weight low osmolality carbohydrate/dose. In one example, the composition comprises about 40 mg of high molecular weight low osmolality carbohydrate/dose. In one example, the composition comprises about 45 mg of high molecular weight low osmolality carbohydrate/dose.

In one example, the high molecular weight low osmolality carbohydrate is dextrin.

Dextrin is a generic term applied to a variety of products obtained by heating a starch in the presence of small amounts of moisture and an acid. Dextrins are a group of low-molecular-weight carbohydrates produced by the hydrolysis of starch or glycogen. “Dextrin” refers to a glucose polymer produced by the hydrolysis of starch (or glycogen) which comprises glucose units linked together by means mainly of α-1,4 linkages. In addition to α-1,4 linkages, there may be a proportion of α-1,6 linkages in a particular dextrin, the amount depending on the starch starting material. Since the rate of biodegradability of α-1,6 linkages is typically less than that for α-1,4 linkages, it is preferred that, for many applications, the percentage of α-1,6 linkages is less than 10% and more preferably less than 5%. In some examples, dextrins are thus mixtures of polymers of D-glucose units linked by alpha-(1->4) or alpha-(1->6) glycosidic bonds.

Dextrins can be produced from starch using enzymes like amylases, as during digestion in the human body and during malting and mashing, or by applying dry heat under acidic conditions (pyrolysis or roasting). The latter process is used industrially. Dextrins produced by heat are also known as pyrodextrins. Typically, dextrins are produced by the hydrolysis of starch obtained from various natural products such as wheat, rice, corn, maize and tapioca.

Dextrins are typically white, yellow, or brown powder that are partially or fully water-soluble, yielding optically active solutions of low viscosity. Dextrins are available from several suppliers and can be readily sourced by the skilled person. The skilled person would readily be able to identify suitable dextrins for use in the context of the present invention. The skilled person would readily be able to detect the presence of dextrin in a substance (e.g. in a composition described herein) using methods known in the art. For example, most dextrins can be detected with iodine solution.

The term “dextrin” encompasses pyrodextrins, digestible dextrins, and hydrogenated products thereof, including derivatives thereof. The term “dextrin derivative” herein means those obtained by chemically or enzymatically processing dextrins, and encompasses, for example, branched dextrins obtained by causing a glycosyltransferase to act on a dextrin, and cyclodextrins obtained by causing a cyclodextrin producing enzyme to act on a starch, in addition to the above-described polydextrose.

In some examples, dextrin is enzymatically processed.

In one example, the dextrin, and thus, the high molecular weight low osmolality carbohydrate, is cluster dextrin (also referred to as highly branched cyclic dextrin). Cluster dextrin is a maltodextrin which has a high molecular weight, but narrow weight distribution, is soluble, and its osmotic pressure is near zero. Typical macromolecular carbohydrates are less soluble than Cluster Dextrin. On the other hand low molecular carbohydrates exhibit higher osmotic pressure, as does pure glucose solution. Along with other digestible contents typically found in sports drinks, this slows their descent to the small intestine. Cluster Dextrin is faster to the small intestine and faster to ramp-up endurance. Solubility is at the heart of Cluster Dextrin's functioning. High molecular weight Cluster Dextrin also degrades slowly, balancing insulin secretion with lipid breakdown. Cluster dextrin is manufactured by Glico Nutrition.

In some examples, the dextrin, and thus, the high molecular weight low osmolality carbohydrate is derived from wheat or corn (e.g. in some examples the dextrin is wheat dextrin or corn dextrin).

In some examples, the dextrin is enzymatically processed wheat or corn.

In one example, the dextrin is derived from wheat (e.g. wheat dextrin). For instance, the dextrin may be a wheat dextrin powder (such as Surbex Nutri-Fiber Wheat Dextrin Powder which is a soluble non viscous fiber).

In another example, the dextrin is derived from con (e.g. coin dextrin).

In a particular example, the dextrin is hydrolysed coin dextrin (e.g. Vitargo).

In some examples, dextrin is present in the composition described herein at an appropriate concentration or amount.

In one example, the composition comprises about 0.5% w/w to about 5% w/w of dextrin.

In another example, the composition comprises about 0.5% w/w to about 3% w/w of dextrin. In another example, the composition comprises about 0.5% w/w to about 2% w/w of dextrin.

In one example, the composition comprises at least about 0.5% w/w of dextrin.

In another example, the composition comprises at least about 1% w/w of dextrin. In another example, the composition comprises at least about 1.5% w/w of dextrin. In a further example, the composition comprises at least about 2% w/w of dextrin. In another example, the composition comprises at least about 2.5% w/w of dextrin. In another example, the composition comprises at least about 3% w/w of dextrin. In another example, the composition comprises at least about 3.5% w/w of dextrin. In a further example, the composition comprises at least about 4% w/w of dextrin. In another example, the composition comprises at least about 4.5% w/w of dextrin. In a further example, the composition comprises at least about 5% w/w of dextrin.

In one example, the composition comprises about 0.5% w/w of dextrin.

In another example, the composition comprises about 1% w/w of dextrin. In another example, the composition comprises about 1.5% w/w of dextrin. In a further example, the composition comprises about 2% w/w of dextrin. In another example, the composition comprises about 2.5% w/w of dextrin. In another example, the composition comprises about 3% w/w of dextrin. In another example, the composition comprises about 3.5% w/w of dextrin.

In a further example, the composition comprises about 4% w/w of dextrin. In another example, the composition comprises about 4.5% w/w of dextrin. In a further example, the composition comprises about 5% w/w of dextrin.

In one example, the composition comprises about 2 mg to about 50 mg of dextrin/dose.

In one example, the composition comprises at least about 2 mg of dextrin/dose.

In one example, the composition comprises at least about 4 mg of dextrin/dose.

In one example, the composition comprises at least about 5 mg of dextrin/dose. In one example, the composition comprises at least about 10 mg of dextrin/dose. In one example, the composition comprises at least about 15 mg of dextrin/dose. In one example, the composition comprises at least about 20 mg of dextrin/dose. In one example, the composition comprises at least about 25 mg of dextrin/dose. In one example, the composition comprises at least about 30 mg of dextrin/dose. In one example, the composition comprises at least about 35 mg of dextrin/dose. In one example, the composition comprises at least about 40 mg of dextrin/dose. In one example, the composition comprises at least about 45 mg of dextrin/dose.

In one example, the composition comprises about 2 mg of dextrin/dose.

In one example, the composition comprises about 4 mg of dextrin/dose.

In one example, the composition comprises about 10 mg of dextrin/dose. In one example, the composition comprises about 15 mg of dextrin/dose. In one example, the composition comprises about 20 mg of dextrin/dose. In one example, the composition comprises about 25 mg of dextrin/dose. In one example, the composition comprises about 30 mg of dextrin/dose. In one example, the composition comprises about 35 mg of dextrin/dose. In one example, the composition comprises about 40 mg of dextrin/dose. In one example, the composition comprises about 45 mg of dextrin/dose.

In one example, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) described herein comprises L-cysteine and dextrin. L-cysteine and dextrin may be present in the composition described herein at any appropriate concentration. Appropriate concentrations or amounts of L-cysteine and dextrin are described elsewhere herein and apply equally to a composition described herein comprising both L-cysteine and dextrin. Illustrative examples of appropriate concentrations or amounts are provided below.

In one example, the composition comprises about 0.5% w/w to 5% w/w of dextrin and about 10% w/w of L-cysteine to about 40% w/w of L-cysteine. In each of these examples, the composition may also comprise about 50% w/w to about 90% w/w of rice bran (e.g. fermented rice bran) and/or about 10,000 cfu/g of bacteria of the Bacillus genus to about 15,000 cfu/g of bacteria of the Bacillus genus (e.g. B. subtilis and/or B. coagulans).

In one example, the composition described herein comprises at least about 0.5% w/w of dextrin and at least about 10% w/w of L-cysteine. For example, the composition described herein may comprise about 0.5% w/w of dextrin and about 10% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 0.5% w/w of dextrin and at least about 20% w/w of L-cysteine. For example, the composition described herein may comprise about 0.5% w/w of dextrin and about 20% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 0.5% w/w of dextrin and at least about 20% w/w of L-cysteine. For example, the composition described herein may comprise about 0.5% w/w of dextrin and about 20% w/w of L-cysteine. In each of these examples, the composition may also comprise at least about 73% w/w of rice bran (e.g. at least about 73% w/w of fermented rice bran) and/or at least 100,000 cfu/g of bacteria of the Bacillus genus (e.g. B. subtilis and/or B. coagulans). For instance, in each of these examples, the composition may comprise about 73% w/w of rice bran (e.g. about 73% w/w of fermented rice bran) and/or 100,000 cfu/g of bacteria of the Bacillus genus (e.g. B. subtilis and/or B. coagulans).

In one example, the composition described herein comprises at least about 0.5% w/w of dextrin and at least about 30% w/w of L-cysteine. For example, the composition described herein may comprise about 0.5% w/w of dextrin and about 30% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 5% w/w of dextrin and at least about 20% w/w of L-cysteine. For example, the composition described herein may comprise about 5% w/w of dextrin and about 20% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 3% w/w of dextrin and at least about 30% w/w of L-cysteine. For example, the composition described herein may comprise about 3% w/w of dextrin and about 30% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition comprises about 2 mg to about 50 mg of dextrin/dose and about 38 mg to about 200 mg of L-cysteine/dose. In each of these examples, the composition may also comprise about 300 mg to about 600 mg of rice bran/dose (e.g. fermented rice bran/dose) and/or about 5,000 cfu of bacteria of the Bacillus genus/dose to about 1×10⁸ cfu of bacteria of the Bacillus genus/dose (e.g. B. subtilis and/or B. coagulans).

In one example, the composition described herein comprises at least about 2 mg of dextrin/dose and at least about 38 mg of L-cysteine/dose. For example, the composition described herein may comprise about 2 mg of dextrin/dose and about 38 mg of L-cysteine/dose. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 2 mg of dextrin and at least about 76 mg of L-cysteine/dose. For example, the composition described herein may comprise about 2 mg of dextrin/dose and about 76 mg of L-cysteine/dose. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 2 mg of dextrin/dose and at least about 114 mg of L-cysteine/dose. For example, the composition described herein may comprise about 2 mg of dextrin/dose and about 114 mg of L-cysteine/dose. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 20 mg of dextrin/dose and at least about 76 mg of L-cysteine/dose. For example, the composition described herein may comprise about 20 mg of dextrin/dose and about 76 mg of L-cysteine/dose. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 4 mg of dextrin/dose and at least about 150 mg of L-cysteine/dose. For example, the composition described herein may comprise about 4 mg of dextrin/dose and about 150 mg of L-cysteine/dose. In each of these examples, the composition may also comprise at least about 552 mg of rice bran/dose (e.g. at least about 552 mg of fermented rice bran/dose) and/or at least 1×10⁵ cfu of bacteria of the Bacillus genus/dose (e.g. B. subtilis and/or B. coagulans). For instance, in each of these examples, the composition may comprise about 552 mg of rice bran/dose (e.g. about 552 mg of fermented rice bran/dose) and/or 1×10⁵ cfu of bacteria of the Bacillus genus/dose (e.g. B. subtilis and/or B. coagulans).

As discussed elsewhere herein, it is suggested that AB001 has four glucose-controlling actions. In one example, without wishing to be bound by theory, it is suggested that upon resuscitation of Bacillus cells and their endospores in duodenum and in the small intestine, the cells scan the biochemical conditions of their micro-environment and start to excrete a unique selection of bio-active substances to optimise the conditions, e.g. pH, conductivity, electrolytes, for their survival and multiplication. Nutrients and substrates are essential for survival and subsequent multiplication. When glucose and other carbohydrates are present in the micro-environment, carbohydrate (e.g. glucose)-targeted enzymes are excreted to advantageously break down glucose as described elsewhere herein.

As stated elsewhere herein, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) described herein may include a cereal (e.g. a cereal grain component such as rice bran). A cereal is any grass cultivated (grown) for the edible components of its grain (botanically, a type of fruit called a caryopsis), composed of endosperm, germ, and bran. The term cereal may also refer to the resulting grain itself (specifically “cereal grain”). Cereal grains are the seeds that come from grasses such as wheat, millet, rice, barley, oats, rye, triticale, sorghum, and maize (corn).

In some examples, the composition for use according to the present invention may include a cereal (e.g. a cereal grain) selected from the group consisting of: wheat, millet, rice, barley, oats, rye, triticale, sorghum, and maize (corn). A person of skill in the art would readily be able to identify a suitable cereal (e.g. cereal grain) for use in the composition described herein.

Bran, also known as miller's bran, is the hard outer layers of cereal grain. It comprises aleurone and pericarp. Corn (maize) bran also includes the pedicel (tip cap). Along with germ, bran is an integral part of whole grains, and is often produced as a byproduct of milling in the production of refined grains. Bran is present in cereal grain, including rice, corn (maize), wheat, oats, barley, rye and millet. Accordingly, in some examples, the composition for use according to the present invention may comprise cereal bran. In some examples, the composition for use according to the present invention may comprise a cereal bran selected from the group consisting of: rice bran, corn (maize) bran, wheat bran, oat bran, barley bran, rye bran and millet bran.

In some examples, the composition for use according to the present invention may comprise oat bran.

In some examples, the composition for use according to the present invention may comprise rice bran.

Rice bran is a by-product of the rice milling process. Generally rice milling yields about 15% w/w broken kernels, about 10% w/w rice bran, about 20% hulls and about 55% w/w whole kernels. The composition of rice bran (in percent by weight) is generally 11-13% of water, 18-21% of crude fat and oil, 14-16% crude protein, 8-10% of crude fiber, 9-12% of ash and 33-36% of carbohydrate. Rice bran has naturally occurring lipases that hydrolyze the oil into glycerol and free fatty acids which give the product a rancid smell and taste. As used herein, “rice bran” refers to the hard outer layer of rice which comprises aleurone and pericarp. Along with germ, it is an integral part of whole rice, and, as mentioned above, is often produced as a by-product of milling in the production of refined rice.

The composition for used according to the present invention described herein may comprise rice bran in any suitable form. Suitable forms include raw rice bran, recently milled (unhydrolyzed) full-fat rice bran, low-fat rice bran, defatted rice bran, fermented rice bran, stabilized rice bran and so forth. Raw rice bran is rice bran as obtained after milling. Low-fat rice bran and defatted rice bran are derived from full-fat rice bran by solvent extraction or the like. Full-fat rice bran has a fat content of about 14-18% by weight and low fat and defatted rice bran have about 3-14% and less than 3% fat, respectively, on a weight basis.

In one example, the rice bran is formulated as raw rice bran, recently milled (unhydrolyzed) full-fat rice bran, low-fat rice bran, defatted rice bran, fermented rice bran and/or stabilized rice bran.

In another, more preferable, example, the rice bran is formulated as fermented rice bran and/or stabilized rice bran.

In a further example, the rice bran is fermented rice bran. Rice bran undergoes a natural/spontaneous fermentation process by naturally occurring microbial strains, typically soil-bound strains, e.g. Bacillus and Pediococcus.

As used herein, “fermented rice bran” refers to rice bran which has undergone a fermentation process. Fermented rice bran contains probiotic microbes that stabilise the microbiome of the small intestine.

Generally, “microbiota” and “microbiome” are used to described microflora.

As used herein “stabilized rice bran” refers to rice bran which has been heated for a short period of time, for example by passing it through a high temperature high pressure extruder. The heat stabilizes the rice bran. In other words, “stabilized rice bran” is thus heat treated rice bran. For instance, rice bran may be stabilized after milling is being by heating it at 130 degrees Celsius for less than 10 seconds. In some examples, stabilized rice bran is a dietary fibre. Typically, stabilized rice bran is a dietary fibre that can be catabolized in the colon. As a dietary fibre, stabilised rice bran is a prebiotic interacting with the microbiome of colon.

The microbial contents of fermented rice bran may have a probiotic and stabilising effect on the microbiome of the small intestine and rice bran, being a dietary fibre, may stabilise the conditions of colon. Another mechanism of action maybe a reduction of intestinal oxidative stress, which normalizes the barrier function of the intestinal mucosa.

Rice bran (e.g. fermented rice bran) is present in the composition described herein in an appropriate amount or concentration as described below. The amount or concentration of rice bran may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of rice bran over the plurality of dosage units.

In one example, the composition comprises about 50% w/w to about 90% w/w of rice bran.

In another example, the composition comprises about 60% w/w to about 80% w/w of rice bran. In another example, the composition comprises about 73% w/w to about 79% w/w of rice bran.

In one example, the composition comprises at least about 50% w/w of rice bran. In another example, the composition comprises at least about 55% w/w of rice bran. In one example, the composition comprises at least about 60% w/w of rice bran. In a further example, the composition comprises at least about 65% w/w of rice bran. In one example, the composition comprises at least about 73% w/w of rice bran. In one example, the composition comprises at least about 75% w/w of rice bran. In one example, the composition comprises at least about 79% w/w of rice bran. In a further example, the composition comprises at least about 85% w/w of rice bran. In one example, the composition comprises at least about 90% w/w of rice bran.

In one example, the composition comprises about 50% w/w of rice bran. In another example, the composition comprises about 55% w/w of rice bran. In one example, the composition comprises about 60% w/w of rice bran. In a further example, the composition comprises about 65% w/w of rice bran. In one example, the composition comprises about 73% w/w of rice bran. In one example, the composition comprises about 75% w/w of rice bran. In one example, the composition comprises about 79% w/w of rice bran. In a further example, the composition comprises about 85% w/w of rice bran. In one example, the composition comprises about 90% w/w of rice bran.

In a particular example, the composition comprises at least about 67% w/w of rice bran (e.g. fermented rice bran). For instance, the composition may comprise about 67% w/w of rice bran (e.g. fermented rice).

In a particular example, the composition comprises at least about 73% w/w of rice bran (e.g. fermented rice). For instance, the composition may comprise about 73% w/w of rice bran (e.g. fermented rice).

In another example, the composition comprises at least about 79% w/w of rice bran (e.g. fermented rice). For instance, the composition may comprise about 79% w/w of rice bran (e.g. fermented rice).

In another example, the composition comprises at least about 79.5% w/w of rice bran (e.g. fermented rice). For instance, the composition may comprise about 79.5% w/w of rice bran (e.g. fermented rice).

In one example, the composition comprises at least about 300 mg of rice bran (e.g. fermented rice bran) per dose. As described elsewhere herein, a dose may be formulated as two capsules.

In one example, the composition comprises at least about 300 mg of rice bran (e.g. fermented rice bran) per dose. In one example, the composition comprises at least about 350 mg of rice bran (e.g. fermented rice bran) per dose. In one example, the composition comprises at least about 400 mg of rice bran (e.g. fermented rice bran) per dose. In one example, the composition comprises at least about 450 mg of rice bran (e.g. fermented rice bran) per dose. In one example, the composition comprises at least about 500 mg of rice bran (e.g. fermented rice bran) per dose. In one example, the composition comprises at least about 550 mg (e.g. at least about 552 mg) of rice bran (e.g. fermented rice bran) per dose.

In one example, the composition comprises about 300 mg of rice bran (e.g. fermented rice bran) per dose. In one example, the composition comprises about 350 mg of rice bran (e.g. fermented rice bran) per dose. In one example, the composition comprises about 400 mg of rice bran (e.g. fermented rice bran) per dose. In one example, the composition comprises about 450 mg of rice bran (e.g. fermented rice bran) per dose. In one example, the composition comprises about 500 mg of rice bran (e.g. fermented rice bran) per dose. In one example, the composition comprises about 550 mg (e.g. about 552 mg) of rice bran (e.g. fermented rice bran) per dose.

As discussed elsewhere herein, a composition comprising one or more bacterial species of the Bacillus genus (e.g. B subtilis and/or B. coagulans), rice bran (e.g. fermented rice bran), L-cysteine and a high molecular weight low osmolality carbohydrate (e.g. dextrin) for use according to the present invention (e.g. for treating and/or preventing type 2 diabetes, pre-diabetes, and/or a symptom thereof in a subject and/or in a method of treating and/or preventing type 2 diabetes, pre-diabetes, and/or a symptom thereof in a subject) is provided. The one or more bacterial species of the Bacillus genus (e.g. B subtilis and/or B. coagulans), rice bran (e.g. fermented rice bran), L-cysteine and a high molecular weight low osmolality carbohydrate (e.g. dextrin) may be present in the composition for use according to the present invention at any appropriate amount or concentration. Appropriate amounts and concentrations of the one or more bacterial species of the Bacillus genus (e.g. B subtilis and/or B. coagulans), rice bran (e.g. fermented rice bran), L-cysteine and the high molecular weight low osmolality carbohydrate (e.g. dextrin) are described elsewhere herein. Illustrative examples of appropriate concentrations are provided below.

In one example, the composition comprises about 0.5% w/w to 5% w/w of dextrin, about 10% w/w of L-cysteine to about 40% w/w of L-cysteine, about 50% w/w to about 90% w/w of rice bran (e.g. fermented rice bran) and about 10,000 cfu/g of bacteria of the Bacillus genus to about 15,000 cfu/g of bacteria of the Bacillus genus (e.g. B. subtilis and/or B. coagulans).

In one example, the composition described herein comprises at least about 0.5% w/w of dextrin, at least about 10% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 0.5% w/w of dextrin, at least about 20% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 0.5% w/w of dextrin, at least about 20% w/w of L-cysteine, at least about 73% w/w of rice bran (e.g. at least about 73% w/w of fermented rice bran) and at least 100,000 cfu/g of bacteria of the Bacillus genus (e.g. B. subtilis and/or B. coagulans).

In one example, the composition described herein comprises at least about 0.5% w/w of dextrin, at least about 30% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 5% w/w of dextrin, at least about 20% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 3% w/w of dextrin, at least about 30% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition comprises about 2 mg to about 50 mg of dextrin/dose, about 38 mg to about 200 mg of L-cysteine/dose, about 300 mg to about 600 mg of rice bran/dose (e.g. fermented rice bran/dose) and about 5,000 cfu of bacteria of the Bacillus genus/dose to about 1×10⁸ cfu of bacteria of the Bacillus genus/dose (e.g. B. subtilis and/or B. coagulans).

In one example, the composition described herein comprises at least about 2 mg of dextrin/dose, at least about 38 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 2 mg of dextrin, at least about 76 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 2 mg of dextrin/dose, at least about 114 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 20 mg of dextrin/dose, at least about 76 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 4 mg of dextrin/dose, at least about 150 mg of L-cysteine/dose, at least about 552 mg of rice bran/dose (e.g. at least about 552 mg of fermented rice bran/dose) and at least 1×10⁵ cfu of bacteria of the Bacillus genus/dose (e.g. B. subtilis and/or B. coagulans).

In a particular example, the composition described herein comprises at least about 0.5% w/w of dextrin, at least about 20% w/w of L-cysteine and at least about 73% w/w of rice bran (e.g. at least about 73% w/w of fermented rice bran). For instance, the composition described herein may comprise about 0.5% w/w of dextrin, about 20% w/w of L-cysteine, and about 73% w/w of rice bran (e.g. about 73% w/w of fermented rice bran). In these examples, fermented rice bran may include one or more bacterial species of the Bacillus genus.

In a particular example the composition described herein comprises about 79.5% w/w fermented rice bran (including one or more bacterial species of the Bacillus genus), about 0.5% w/w dextrin and about 20.0% w/w L-cysteine.

The composition for use according to the present invention (e.g. (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) described herein may include one or more additional components e.g. one or more additional ingredients. For example, the composition described herein may further comprise emulsifying agents, filling agents, excipients and/or non-active ingredients. A person of skill in the art would readily understand what is meant by “emulsifying agent”, “filling agent”, “excipients” and “non-active ingredient” in the context of the compositions for use according to the present invention provided herein. Non-limiting examples of additional components include vitamin B12, fatty acid magnesium salts (e.g. magnesium stearate), calcium phosphate, potassium phosphate, silicon dioxide and cellulose (e.g. microcrystalline cellulose).

The additional components described herein may be in any suitable form. Suitable forms would be readily identifiable by a person skilled in the art.

Accordingly, in one example, the composition described herein may further comprise one or more of: vitamin B12, a fatty acid magnesium salt (e.g. magnesium stearate), calcium phosphate, potassium phosphate, silicon dioxide and cellulose (e.g. microcrystalline cellulose). In another example, the composition described herein may further comprise two or more of: vitamin B12, a fatty acid magnesium salt (e.g. magnesium stearate), calcium phosphate, potassium phosphate, silicon dioxide and cellulose (e.g. microcrystalline cellulose). In another example, the composition described herein may further comprise three or more of: vitamin B12, a fatty acid magnesium salt (e.g. magnesium stearate), calcium phosphate, potassium phosphate, silicon dioxide and cellulose (e.g. microcrystalline cellulose). In another example, the composition described herein may further comprise four or more of: vitamin B12, a fatty acid magnesium salt (e.g. magnesium stearate), calcium phosphate, potassium phosphate, silicon dioxide and cellulose (e.g. microcrystalline cellulose).

In one example, the composition described further comprises vitamin B12, a fatty acid magnesium salt (e.g. magnesium stearate), calcium phosphate, potassium phosphate, silicon dioxide and cellulose (e.g. microcrystalline cellulose).

Examples of fatty acid magnesium salts include magnesium stearate. Accordingly, in one example, the composition described herein may further comprise magnesium stearate. Magnesium stearate is the magnesium salt of the fatty acid stearic acid. Magnesiums salts of fatty acids (e.g. magnesium stearate) may be an excipient, an inactive ingredient and/or may be used as a lubricant for manufacturing machines. Magnesium stearate is a GRAS-listed ingredient.

In some examples, vitamin B12 is added for regulatory purposes.

In some examples, magnesium salts (e.g. magnesium stearate), calcium salts and/or potassium salts are non-active ingredients (e.g. they have no clinical effects). For example, magnesium salts (e.g. magnesium stearate), calcium salts and/or potassium salts may be added as filling aids (e.g. filling agents) which help capsule filling machines work effectively.

In some examples, a chromium salt is added for regulatory purposes. For example, the chromium salt may be chromium chloride. Any appropriate amount of chromium salt (e.g. chromium chloride) may be used.

In one example, the composition described herein comprises at least about 20 mcg of chromium per dose. In one example, the composition described herein comprises about 20 mcg of chromium per dose.

In one example, the composition described herein comprises about 80 mcg to about 100 mcg of CrCl3 (chromium chloride) per dose.

In one example, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) described herein further comprises microcrystalline cellulose. Microcrystalline cellulose may be used as an emulsifying agent, filling aid and/or a non-active ingredient. Microcrystalline cellulose (and also maltodextrin) may be used as a cake-forming excipient when punching tablets and as a fluidity agent when making capsule formulations. In some examples the composition described herein further comprises maltodextrin.

In one example, the composition comprises about 5% w/w to about 50% w/w of microcrystalline cellulose. In another example, the composition comprises about 5% w/w to about 35% w/w of microcrystalline cellulose. In another example, the composition comprises about 5% w/w to about 20% w/w of microcrystalline cellulose. In another example, the composition comprises about 5% w/w to about 15% w/w of microcrystalline cellulose. In another example, the composition comprises about 5% w/w to about 8% w/w of microcrystalline cellulose.

In one example, the composition comprises at least about 5% w/w of microcrystalline cellulose.

In one example, the composition comprises at least about 8% w/w of microcrystalline cellulose.

In another example, the composition comprises at least about 10% w/w of microcrystalline cellulose. In another example, the composition comprises at least about 15% w/w of microcrystalline cellulose. In a further example, the composition comprises at least about 20% w/w of microcrystalline cellulose. In another example, the composition comprises at least about 25% w/w of microcrystalline cellulose. In another example, the composition comprises at least about 30% w/w of microcrystalline cellulose. In yet a further example, the composition comprises at least about 35% w/w of microcrystalline cellulose. In another example, the composition comprises at least about 40% w/w of microcrystalline cellulose. In another example, the composition comprises at least about 45% w/w of microcrystalline cellulose. In another example, the composition comprises at least about 50% w/w of microcrystalline cellulose.

In one example, the composition comprises about 5% w/w of microcrystalline cellulose.

In one example, the composition comprises about 8% w/w of microcrystalline cellulose.

In another example, the composition comprises about 10% w/w of microcrystalline cellulose. In another example, the composition comprises about 15% w/w of microcrystalline cellulose. In a further example, the composition comprises about 20% w/w of microcrystalline cellulose. In another example, the composition comprises about 25% w/w of microcrystalline cellulose. In yet a further example, the composition comprises about 30% w/w of microcrystalline cellulose. In another example, the composition comprises about 35% w/w of microcrystalline cellulose. In another example, the composition comprises about 40% w/w of microcrystalline cellulose. In another example, the composition comprises about 45% w/w of microcrystalline cellulose. In another example, the composition comprises about 50% w/w of microcrystalline cellulose.

In one example, the composition described herein comprises at least about 30 mg of microcrystalline cellulose per dose. As described elsewhere herein, a dose may be formulated in two capsules.

In one example, the composition described herein comprises at least about 40 mg of microcrystalline cellulose per dose.

In one example, the composition described herein comprises at least about 50 mg of microcrystalline cellulose per dose. In one example, the composition described herein comprises at least about 60 mg of microcrystalline cellulose per dose. In one example, the composition described herein comprises at least about 70 mg of microcrystalline cellulose per dose. In one example, the composition described herein comprises at least about 80 mg of microcrystalline cellulose per dose. In one example, the composition described herein comprises at least about 90 mg of microcrystalline cellulose per dose. In one example, the composition described herein comprises at least about 100 mg of microcrystalline cellulose per dose.

In one example, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom) described herein further comprises magnesium stearate. Magnesium stearate may be used as an emulsifying agent, filling aid and/or a non-active ingredient.

In one example, the composition comprises about 0.2% w/w to about 1.5% w/w of magnesium stearate. In another example, the composition comprises about 0.5% w/w to about 1.4% w/w of magnesium stearate. In another example, the composition comprises about 0.6% w/w to about 1.3% w/w of magnesium stearate.

In one example, the composition comprises at least about 0.2% w/w of magnesium stearate. In another example, the composition comprises at least about 0.3% w/w of magnesium stearate. In another example, the composition comprises at least about 0.4% w/w of magnesium stearate. In a further example, the composition comprises at least about 0.5% w/w of magnesium stearate.

In another example, the composition comprises at least about 0.6% w/w of magnesium stearate.

In another example, the composition comprises at least about 0.7% w/w of magnesium stearate. In yet a further example, the composition comprises at least about 0.8% w/w of magnesium stearate. In another example, the composition comprises at least about 0.9% w/w of magnesium stearate.

In another example, the composition comprises at least about 1.0% w/w of magnesium stearate.

In another example, the composition comprises at least about 1.1% w/w of magnesium stearate. In another example, the composition comprises at least about 1.2% w/w of magnesium stearate. In another example, the composition comprises at least about 1.3% w/w of magnesium stearate. In another example, the composition comprises at least about 1.4% w/w of magnesium stearate. In another example, the composition comprises at least about 1.5% w/w of magnesium stearate.

In one example, the composition comprises about 0.2% w/w of magnesium stearate. In another example, the composition comprises about 0.3% w/w of magnesium stearate. In another example, the composition comprises about 0.4% w/w of magnesium stearate. In a further example, the composition comprises about 0.5% w/w of magnesium stearate.

In another example, the composition comprises about 0.6% w/w of magnesium stearate.

In another example, the composition comprises about 0.7% w/w of magnesium stearate. In yet a further example, the composition comprises about 0.8% w/w of magnesium stearate. In another example, the composition comprises about 0.9% w/w of magnesium stearate.

In another example, the composition comprises about 1.0% w/w of magnesium stearate.

In another example, the composition comprises about 1.1% w/w of magnesium stearate. In another example, the composition comprises about 1.2% w/w of magnesium stearate. In another example, the composition comprises about 1.3% w/w of magnesium stearate. In another example, the composition comprises about 1.4% w/w of magnesium stearate. In another example, the composition comprises about 1.5% w/w of magnesium stearate.

In one example, the composition described herein comprises at least about 4 mg of magnesium stearate per dose. As described elsewhere herein, a dose may be formulated in two capsules.

In one example, the composition described herein comprises at least about 5 mg of magnesium stearate per dose.

In one example, the composition described herein comprises at least about 6 mg of magnesium stearate per dose. In one example, the composition described herein comprises at least about 7 mg of magnesium stearate per dose. In one example, the composition described herein comprises at least about 8 mg of magnesium stearate per dose. In one example, the composition described herein comprises at least about 9 mg of magnesium stearate per dose. In one example, the composition described herein comprises at least about 10 mg of magnesium stearate per dose.

In one example, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom) described herein further comprises silicon dioxide. Silicon dioxide may be used as an emulsifying agent, filling aid and/or a non-active ingredient.

In one example, the composition comprises about 0.5% w/w to about 4% w/w of silicon dioxide. In another example, the composition comprises about 0.7% w/w to about 3% w/w of silicon dioxide. In another example, the composition comprises about 0.9% w/w to about 2% w/w of silicon dioxide.

In one example, the composition comprises at least about 0.5% w/w of silicon dioxide.

In another example, the composition comprises at least about 1% w/w of silicon dioxide.

In another example, the composition comprises at least about 1.5% w/w of silicon dioxide. In a further example, the composition comprises at least about 2% w/w of silicon dioxide. In another example, the composition comprises at least about 2.5% w/w of silicon dioxide. In another example, the composition comprises at least about 3% w/w of silicon dioxide. In yet a further example, the composition comprises at least about 3.5% w/w of silicon dioxide. In another example, the composition comprises at least about 4% w/w of silicon dioxide.

In one example, the composition comprises about 0.5% w/w of silicon dioxide.

In another example, the composition comprises about 1% w/w of silicon dioxide.

In another example, the composition comprises about 1.5% w/w of silicon dioxide. In a further example, the composition comprises about 2% w/w of silicon dioxide. In another example, the composition comprises about 2.5% w/w of silicon dioxide. In another example, the composition comprises about 3% w/w of silicon dioxide. In yet a further example, the composition comprises about 3.5% w/w of silicon dioxide. In another example, the composition comprises about 4% w/w of silicon dioxide.

In one example, the composition described herein comprises at least about 4 mg of silicon dioxide per dose. As described elsewhere herein, a dose may be formulated in two capsules.

In one example, the composition described herein comprises at least about 5 mg of silicon dioxide per dose. In one example, the composition described herein comprises at least about 6 mg of silicon dioxide per dose. In one example, the composition described herein comprises at least about 7 mg of silicon dioxide per dose. In one example, the composition described herein comprises at least about 8 mg of silicon dioxide per dose.

In one example, the composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom) described herein further comprises vitamin B12. Vitamin B12 may be included in the composition described herein for regulatory purposes. In one example, the composition comprises at least 15% of recommended daily intake (RDI), i.e. 0.38 mcg up to 2.4 mcg (EU) and 2.5 mcg for USA.

In one example, the composition described herein comprises at least about 0.76 μg (mcg) of vitamin B12 per dose. In one example, the composition described herein comprises at least about 0.9 μg (mcg) of vitamin B12 per dose.

The composition may be formulated in any appropriate form. For example, it may be in the form of a tablet or capsule. In one example, the composition is formulated as an acid resistant tablet or capsule. Generally, “capsule” refers to both empty and filled capsules whereas “shell” specifically refers to an empty capsule unless the context requires otherwise.

In some examples, the composition described herein is comprised within a capsule (e.g. within a shell). In some examples, the composition described herein is comprised within an acid resistant capsule (e.g. within an acid resistant shell). As would be clear to a person of skill in the art, when reference is made to a % w/w of an ingredient present within the composition, this does not take into account any weight attributed to the capsule (and thus only takes into account the % w/w of the composition within the capsule).

In one example, the acid resistant tablet or capsule comprises a film coating, wherein the film coating comprises hydroxypropyl methylcellulose (HPMC). HPMC is a semisynthetic, inert, viscoelastic polymer used in various applications. For instance, HPMC may be used as an excipient in oral tablet and capsule formulations, where, depending on the grade, it functions as controlled release agent to delay the release of a medicinal compound into the digestive tract. In tablets, HPMC may also be used as a binder and/or as a component of tablet coatings.

In one example, the composition for use according to the present invention described herein is comprised within a (capsule) shell wherein the shell comprises hydroxypropyl methylcellulose (HPMC).

A person of skill in the art would understand the meaning of “acid resistant” in the context of the present invention, particularly within the context of an ingestible composition as described herein. For instance, the composition described herein may be formulated as an acid resistant tablet or capsule or may be comprised within an acid resistant capsule (e.g. an acid resistant shell) which is dissolved once reaching the duodenum, allowing the microbial cells and spores to be released and settle in the upper part of the intestinal tract.

In one example, the composition is present within a capsule, wherein the capsule comprises about 80 mg to about 100 mg of HPMC. For example, the capsule may comprise at least about 80 mg of HPMC. In one example, the capsule comprises at least about 90 mg of HPMC. In one example, the capsule comprises at least about 100 mg of HPMC. In one example, the capsule comprises about 80 mg of HPMC. In one example, the capsule comprises about 90 mg of HPMC. In another example, the capsule comprises about 100 mg of HPMC.

Typically HPMC is present as a film coating at the exterior surface of the capsule described herein.

The composition described herein may be in unit dosage form. Where the composition described herein is in unit dosage form, it may be that one tablet or capsule is administered and this constitutes a dose, alternatively it may be that two tablets or capsules are administered and this constitutes a dose. Suitable dosages and regimens can be determined by a person skilled in the art, based on the examples below. Accordingly, in one example, a dose of the composition according to the present invention includes a plurality of small tablets or capsules (e.g. two).

The term “dosage form” as used herein, refers to an amount of medication to be taken at one time, optionally in regular intervals. This is also referred to as a “dose” herein.

In an example, the invention provides a solid unit dose form for oral administration.

In a particular example, a dose of the composition described herein comprises: about 300 mg of fermented rice bran, about 38 mg of L-cysteine, about 30 mg of microcrystalline cellulose, about 4 mg of magnesium stearate, about 4 mg of silicon dioxide, about 2 mg of dextrin, and about 5,000 cfu bacteria from the Bacillus genus. Optionally, a dose of the composition described herein may further comprise about 0.76 μg (mcg) vitamin B12 and/or any suitable amount of Chromium Chloride. In this particular example, the dose may be formulated as two tablets or capsules. In other words, the amounts of the components may represent a total amount of the component present in the dose (i.e. in the two tablets or capsules). A skilled person would thus understand that at least each of these components, in at least these amounts, provide an effective dose in accordance with the invention. Accordingly, in one example, dose of the composition described herein comprises: at least about 300 mg of fermented rice bran, at least about 38 mg of L-cysteine, at least about 30 mg of microcrystalline cellulose, at least about 4 mg of magnesium stearate, at least about 4 mg of silicon dioxide, at least about 2 mg of dextrin, and at least about 5,000 cfu bacteria from the Bacillus genus. Optionally, a dose of the composition described herein may further comprise at least about 0.76 μg (mcg) vitamin B12 and/or any suitable amount of Chromium Chloride.

In a particular example, a dose of the composition described herein comprises: about 552 mg of fermented rice bran, about 150 mg of L-cysteine, about 40 mg of microcrystalline cellulose, about 4.8 mg of magnesium stearate, about 4 mg of silicon dioxide, about 4 mg of dextrin, and about 100,000 cfu bacteria from the Bacillus genus. Optionally, a dose of the composition described herein may further comprise about 0.9 μg (mcg) vitamin B12 and/or any suitable amount of Chromium Chloride. In this particular example, the dose may be formulated as two tablets or capsules. In other words, the amounts of the components may represent a total amount of the component present in the dose (i.e. in the two tablets or capsules). A skilled person would thus understand that at least each of these components, in at least these amounts, provide an effective dose in accordance with the invention. Accordingly, in one example, dose of the composition described herein comprises: at least about 552 mg of fermented rice bran, at least about 150 mg of L-cysteine, at least about 40 mg of microcrystalline cellulose, at least about 4.8 mg of magnesium stearate, at least about 4 mg of silicon dioxide, at least about 4 mg of dextrin, and at least about 100,000 cfu bacteria from the Bacillus genus. Optionally, a dose of the composition described herein may further comprise at least about 0.9 μg (mcg) vitamin B12 and/or any suitable amount of Chromium Chloride.

Typically HPMC is present as a film coating at the exterior surface of the capsule described herein.

The composition described herein may be used as (or used as part of) a dietary supplement, a nutraceutical, a food composition, a medical food, or a medicament.

The term “dietary supplement” or “food supplement” as used herein, refers to a composition which is consumed in addition to the daily meals or in between.

The term “food composition” as used herein, refers to any kind of composition which is eatable and/or drinkable without causing toxic symptoms in the subject eating or drinking the respective composition.

The use of a composition described herein for degrading glucose is provided herein. Accordingly, in one example, use of a composition comprising one or more bacterial species of the Bacillus genus, rice bran, L-cysteine and a high molecular weight low osmolality carbohydrate for degrading glucose is provided. The term “degrading glucose” is described elsewhere herein and applies equally here.

The composition may be used for degrading glucose in a subject (e.g. the use may be in vivo). The subject may be any suitable subject, for example, the subject may be human. The subject may be a human that is intending to consume or has consumed a carbohydrate (e.g. glucose).

In one example, the composition may be used for metabolising glucose in the gut of the subject. The term “metabolising glucose” is described elsewhere herein and applies equally here. In one example, the composition may be used for metabolising glucose in the intestine of the subject. In a particular example, the composition may be used for metabolising glucose in the small intestine of the subject. More specifically, the composition may be used for metabolising glucose in the duodenum of the subject.

In one example, the composition may be used for reducing absorption of glucose into the blood of the subject. A person of skill in the art would be able to determine a reduction in absorption of glucose into the blood in the presence of the composition provided herein (compared to when the composition is not used) using methods known in the art.

When the composition is used to degrade glucose in a subject, it may be used for reducing blood glucose concentration in the subject. A person of skill in the art would be able to determine a reduction in blood glucose concentration in the presence of the composition provided herein (compared to when the composition is not used) using methods known in the art.

Use of a composition described herein (e.g. a composition comprising one or more bacterial species of the Bacillus genus, rice bran, L-cysteine and a high molecular weight low osmolality carbohydrate) for controlling blood glucose in a subject is also provided.

Use of a composition described herein (e.g. a composition comprising one or more bacterial species of the Bacillus genus, rice bran, L-cysteine and a high molecular weight low osmolality carbohydrate) for:
(a) reducing the level of one or more of the following in the blood of a subject: HbA1c, glucose (e.g. fasting glucose), insulin, intact pro-insulin and/or hsCRP;
(b) reducing the HOMA-IR score in a subject; and/or
(c) increasing the level of adiponectin in the blood of a subject,
is also provided herein.

“HbA1c”, “glucose” e.g fasting glucose, “insulin, “intact pro-insulin”, “hsCRP”, the “HOMA-IR score” and “adiponectin” are described elsewhere herein and apply equally here.

The subject may be any suitable subject, for example, the subject may be human. The subject may be a human that is intending to consume or has consumed a carbohydrate (e.g. glucose).

In some examples, the compositions for use according to the present invention are in the form of a pharmaceutical formulation, comprising the composition of the invention. As would be clear to a person of skill in the art, a pharmaceutical formulation is a formulation that is suitable for administration to a subject (e.g. for degrading glucose). The pharmaceutical formulation may be for administration to a subject for preventing and/or treating a disease, condition or illness (e.g. for in treating and/or preventing type 2 diabetes, pre-diabetes, and/or a symptom thereof in a subject, and/or for administration in a method of treating and/or preventing type 2 diabetes, pre-diabetes, and/or a symptom thereof in a subject. A pharmaceutical formulation, as used herein, comprises an effective dose of the composition of the invention.

As used herein, a “pharmaceutical composition” refers to a composition having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions or preparations typically meet the microbiological specifications and criteria for non-sterile pharmaceutical products as described in the U.S. Pharmacopeia (USP) or European Pharmacopoeia (EP). Accordingly, the composition described herein may be formulated as a pharmaceutical composition. In some examples, the pharmaceutical composition is non-sterile.

### Methods of Treatments and Uses

As described elsewhere herein, the composition described herein may be used in vitro or in vivo for degrading glucose.

The inventors have surprisingly discovered that a composition comprising one or more bacterial species of the Bacillus genus, rice bran, L-cysteine and a high molecular weight low osmolality carbohydrate may advantageously be used to safely improve the cardiometabolic situation of a subject, in particular a subject having type 2 diabetes and/or pre-diabetes (as exemplified in the examples below). The composition may therefore advantageously provide a valuable supplement to existing treatment combinations in a subject having type 2 diabetes and/or pre-diabetes.

Accordingly, a composition comprising one or more bacterial species of the Bacillus genus, rice bran, L-cysteine and a high molecular weight low osmolality carbohydrate for use in treating and/or preventing type 2 diabetes, pre-diabetes, and/or a symptom thereof in a subject is provided.

A method of treating and/or preventing type 2 diabetes, pre-diabetes, and/or a symptom thereof in a subject, comprising administering a composition comprising one or more bacterial species of the Bacillus genus, rice bran, L-cysteine and a high molecular weight low osmolality carbohydrate to the subject is also provided.

Diabetes mellitus, also commonly known as diabetes, is a metabolic disorder that results in elevation of the blood glucose level because of relative or absolute deficiency in the pancreatic hormone insulin. Insulin is secreted from the pancreas into the blood in response to the blood glucose level and a major function is to direct blood glucose into body stores, whereby the blood glucose level is controlled. Type 2 diabetes mellitus, formerly called adult-onset or noninsulin-dependent diabetes, is a chronic disease marked by perturbations in both glucose and lipid metabolism. Type 2 diabetes is characterized by insulin resistance and impaired beta cell function, which includes impaired first phase insulin release, reduced beta cell pulse mass and insulin deficiency. In other words, in patients having type 2 diabetes, the body is resistant to the effects of insulin and/or doesn't produce enough insulin to maintain a normal glucose level. Individuals with Type 2 diabetes have a combination of increased insulin resistance and decreased insulin secretion that combine to cause hyperglycemia as discussed below. Dyslipidemia is also an integral component of the metabolic perturbations that characterise type 2 diabetes.

Insulin resistance refers to a physiological condition where the natural hormone insulin becomes less effective at lowering blood sugars, for example, because cells are diminished in the ability to respond to the action of insulin in promoting the transport of the sugar glucose from blood into muscles and other tissues (e.g. sensitivity to insulin decreases). Insulin resistance ranges from normal (insulin sensitive) to insulin resistant (IR). In some examples, insulin resistance may mean that insulin is unable of eliciting its normal anabolic responses at maximal dosage of the hormone. An inadequate response to insulin leads to decreased glucose uptake (predominantly in muscle and adipose tissue) and increased hepatic gluconeogenesis, both of which cause circulating blood glucose concentrations to rise. To maintain homeostasis and prevent hyperglycemia (excessive serum glucose levels) pancreatic β-cells increase their insulin secretion, causing hyperinsulinemia (high blood insulin levels). Early in the progression to diabetes but before the development of type 2 diabetes, the pancreas is able to overcome insulin resistance and maintain euglycemia by increasing insulin production. However, later in the progression to diabetes, compensatory insulin secretion by the pancreas fails to overcome the insulin resistance of the body and normal blood (e.g plasma) glucose concentrations can no longer be maintained, thus resulting in hyperglycemia. The symptoms of hyperglycemia are polyurea (passage of a large volume of urine in a given period) and polydipsia (excessive thirst). Chronic hyperglycemia exerts deleterious effects on pancreatic β-cell-function by means of glucose desensitisation (further reduction of insulin sensitivity of the body, e.g. increased insulin resistance) and exhaustion and apoptosis of β-cells, which impairs insulin secretion. This will ultimately result in overt type 2 diabetes, typically characterized by a fasting venous whole blood glucose concentration of over 7.0 mmol/I (126 mg/dL).

The term “type 2 diabetes”, “T2D” or “TIID”, are well defined in the art. Similarly, the term “insulin resistance” is well defined in the art.

Type 2 diabetes coincides with a marked decrease in life expectancy and is a disproportionately expensive disease that requires long-term medical attention in order to limit the development of short- and long-term complications associated with the disease. These complications include hyperinsulinemia, hyperglycemia, hypoglycemia (serum glucose <50 mg/dL), ketoacidosis, increased risk of infections, microvascular complications (e.g., retinopathy, nephropathy), neuropathic complications, and macrovascular disease such as cardiovascular disease (CVD) due to severe arteriosclerosis. The morbidity and mortality associated with diabetes is primarily caused by these complications. For instance, diabetes is the major cause of blindness, as well as an important cause of lower-limb amputation and renal disease.

Symptoms of type 2 diabetes are well known to persons of skill in the art. Non-limiting examples of symptoms of type 2 diabetes include increased thirst, frequent urination, increased hunger, unintended weight loss, fatigue, blurred vision, slow-healing sores, frequent infections, numbness or tingling in the hands or feet, and areas of darkened skin, usually in the armpits and neck.

As used herein “symptoms” refer to a presentation that is indicative of a disease, condition, or illness. In other words, a symptoms refers to a phenotype associated with disease, condition or illness that can be used to facilitate diagnosis of the disease, condition or illness. In some contexts, a combination of symptoms may be needed to facilitate diagnosis of a disease, condition or illness.

“Pre-diabetes” refers to one or more early diabetes-related conditions including impaired glucose utilization, abnormal or impaired fasting glucose levels, impaired glucose tolerance, impaired insulin sensitivity and insulin resistance. Prediabetes is associated with the simultaneous presence of insulin resistance and β-cell dysfunction, abnormalities that start before glucose changes are detectable. Prediabetes is typically characterised by blood glucose levels above normal but below diabetes thresholds, it defines a high chance of developing diabetes.

Symptoms of pre-diabetes include, but are not limited to, increased thirst, frequent urination, increased hunger, unintended weight loss, fatigue, blurred vision, slow-healing sores, frequent infections, numbness or tingling in the hands or feet, genital itching or thrush and areas of darkened skin, usually in the armpits and neck.

Symptoms of type 2 diabetes and/or pre-diabetes may also include insulin resistance, β-cell dysfunction, unregulated glycemic control, chronic systemic inflammation and/or intestinal inflammation, hyperinsulinemia, hyperglycemia, hypoglycemia (serum glucose <50 mg/dL), ketoacidosis, increased risk of infections, microvascular complications (i.e., retinopathy, nephropathy), neuropathic complications, and macrovascular disease such as cardiovascular disease (CVD) due to severe arteriosclerosis).

Advantageously, composition for use according to the invention may be used to improve the above mentioned symptoms of type 2 diabetes and pre-diabetes. The inventors have specifically demonstrated that the composition described herein (AB001) may be used to stabilise glucose levels and/or improve glycemic control, in particular in subjects having type 2 diabetes and/or pre-diabetes. Advantageously, the composition described herein may also be used to reduce insulin resistance, ß-cell dysfunction and/or inflammation (e.g. chronic systemic inflammation and/or intestinal inflammation) in subjects having type 2 diabetes and/or pre-diabetes. In some examples, the inflammation may be intestinal and/or pancreatic inflammation.

Accordingly, administration of the composition to the subject may:
(a) reduce the level of one or more of HbA1c, glucose (e.g. fasting glucose), insulin, intact pro-insulin or hsCRP in the blood;
(b) reduce the HOMA-IR score; and/or
(c) increase the level of adiponectin in the blood;
in subjects having type 2 diabetes and/or a symptom thereof.

Accordingly, administration of the composition to the subject may:
(a) reduce the level of one or more of HbA1c, glucose (e.g. fasting glucose), insulin, intact pro-insulin or hsCRP in the blood;
(b) reduce the HOMA-IR score; and/or
(c) increase the level of adiponectin in the blood;
in subjects having pre-diabetes and/or a symptom thereof.

A person of skill in the art would readily be able to diagnose type 2 diabetes and/or pre-diabetes using routine methods known in the art, for example using reference values known in the art. For example, there are a number of tests which can be used to determine if, e.g., a human subject is affected with pre-diabetes and/or type 2 diabetes. Such tests include, e.g., the A1C test, fasting plasma glucose test (FPG), urine glucose test, random blood glucose tests, and the oral glucose tolerance test (OGTT). Examples of reference values used with these methods are provided below. In some examples, several tests are used in combination to reach a diagnosis.

Type 2 diabetes is usually diagnosed using the glycated hemoglobin (A1C) test. The hemoglobin A1c (glycated hemoglobin, glycosylated hemoglobin, HbA1c, or A1c) test is used to evaluate a person's level of glucose control. The test shows an average of the blood sugar level over the past 90 days and represents a percentage. For example, this blood test may indicate average blood sugar level for the past two to three months. Hemoglobin is a protein only found in red blood cells. Since red blood cells live about an average of three months, the A1c test will reflect those red blood cells that are present in the bloodstream at the time of the test; this is why the A1c serves as an average of blood sugar control. The main job of hemoglobin is to carry oxygen from the lungs to all the cells of the body. Hemoglobin becomes glycated or coated with glucose from the bloodstream. Glucose that is present in the blood will attach to the hemoglobin protein, and increased glucose levels will reflect on the surface of the hemoglobin protein, thereby rendering a higher A1c level. Typically, a blood sample is used for testing (and diagnosis), in particular a whole blood sample or a sample drawn from whole blood (e.g. serum and/or plasma). A person of skill in the art would readily be able to identify a suitable sample type depending on the parameter to be determined. Non-limiting examples of reference values that may be used in this test are as follows: below 5.7% is normal, 5.7% to <6.5% is diagnosed as prediabetes (this indicates impaired glucose regulation), 6.5% or higher on two separate tests indicates diabetes.

Type 2 diabetes and/or pre-diabetes may also be identified using a fasting blood sugar test. The FPG test is a blood test that looks at blood glucose levels after a fast. A fast involves not eating or drinking anything (except for water) for at least eight hours (e.g. before having the test done). Usually, a blood sample is taken after an overnight fast. Prolonged fasting triggers a hormone called glucagon, which is produced by the pancreas and causes the liver to release glucose (blood sugar) into the bloodstream. If you don't have diabetes, your body reacts by producing insulin, which prevents hyperglycemia (high blood sugar). However, if your body cannot generate enough insulin or cannot appropriately respond to insulin, fasting blood sugar levels will stay high. Typically, a blood sample is used for testing (and diagnosis), in particular a whole blood sample or a sample drawn from whole blood (e.g. serum and/or plasma). A person of skill in the art would readily be able to identify a suitable sample type depending on the parameter to be determined. A non-limiting example of how results may be interpreted is as follows: less than 100 mg/dL (5.6 mmol/L) is normal, 100 to <126 mg/dL (5.6 to 6.9 mmol/L) is diagnosed as prediabetes (this indicates impaired fasting glucose/impaired fasting glycemia), 126 mg/dL (7 mmol/L) or higher on two separate tests is diagnosed as diabetes.

Type 2 diabetes and/or pre-diabetes may also be identified using a oral glucose tolerance test. The Glucose Tolerance Test (GTT), also referred to as the Oral Glucose Tolerance Test (OGTT), is a method which can help to diagnose instances of diabetes mellitus or insulin resistance. A glucose tolerance test is used to determine a person's ability to handle a glucose load, it determines how quickly glucose is cleared from the blood. The test can show whether a person can metabolize a standardized measured amount of glucose. This test is less commonly used than the others, except during pregnancy. Before the test one will be asked not to eat, or drink certain fluids, for up to 8 to 12 hours before the test. One may also be asked to not take certain medications in the lead up to the test, but only if these would affect the test results. For the test itself, blood is taken to measure your blood glucose level before the test. The next stage is to consume a very sweet tasting, glucose drink. Further blood samples will then be taken either at regular intervals (30 or 60 minutes), or a single test is taken after 2 hours. Typically, whole blood samples are used for testing (and diagnosis). A non-limiting example of how results may be interpreted is as follows: a fasting value (before test) of under 6 mmol/L and at 2 hours under 7.8 mmol/L is normal, a fasting value (before test) of 6.0 to 7.0 mmol/L and at 2 hours 7.8 to 11.1 mmol/L is diagnosed as pre-diabetes (indicating impaired glucose tolerance), a fasting value (before test) of over 7.0 mmol/L and at 2 hours over 11.1 mmol/L suggests diabetes.

Referring to a normoglycemic (euglycemia) reference value, in some examples, one can use the following cut-off numbers when measuring is performed using the above-mentioned tests: A1C test—under 5.7%; FPG test under 100 mg/dl; and OGTT under 7.8 mmol/L at 2 hours. Furthermore, normoglycemia (euglycemia) may be indicated by interstitial glucose levels of 70-180 mg/dL (where >180 mg/dL indicates hyperglycemia and <70 mg/dL indicates hypoglycemia).

As would be clear to the skilled person, as used herein, “interstitial glucose” refers to glucose in interstitial fluid. Glucose is transferred from the capillary endothelium to interstitial fluid typically by simple diffusion across a concentration gradient without the need of an active transporter. Blood flow to the area dictates the amount of glucose delivered. Generally, interstitial glucose values (e.g. levels) are determined by the rate of glucose diffusion from plasma to the interstitial fluid and the rate of glucose uptake by subcutaneous tissue cells. Interstitial glucose levels may be measured using routine methods known in the art. For example, interstitial glucose levels can be measured using systems for continuous glucose monitoring (CGM) (as used in the examples section below). The glucose sensors of these CGM systems are constructed as needle sensors that are inserted in the subcutaneous adipose tissue to access to interstitial fluid (ISF).

In some examples, the composition is for treating and/or preventing type 2 diabetes and/or a symptom thereof.

Accordingly, in some examples, a composition comprising one or more bacterial species of the Bacillus genus, rice bran, L-cysteine and a high molecular weight low osmolality carbohydrate for use in treating and/or preventing type 2 diabetes and/or a symptom thereof in a subject is provided.

In some examples, a method of treating and/or preventing type 2 diabetes and/or a symptom thereof in a subject, comprising administering a composition comprising one or more bacterial species of the Bacillus genus, rice bran, L-cysteine and a high molecular weight low osmolality carbohydrate to the subject is also provided.

As discussed elsewhere herein, the inventors have surprisingly shown that, in patients with type 2 diabetes, AB001 has beneficial effects vs placebo on glycemic control (as measured by time in normoglycemia, assessed by continuous glucose measurement, and using biomarkers of glycemic control, namely HbA1c and fasting glucose) and on biomarkers of inflammation (e.g. chronic systemic inflammation, assessed using adiponectin or hsCRP as biomarkers), insulin resistance and ß-cell dysfunction (assessed using blood insulin and intact pro-insulin as biomarkers and, the HOMA-IR score). The inventors have thus advantageously demonstrated that the composition described herein (AB001) may be used to stabilise glucose levels and/or improve glycemic control in subjects having type 2 diabetes, pre-diabetes and/or symptoms thereof. Advantageously, the composition described herein may also be used to reduce insulin resistance, ß-cell dysfunction and/or inflammation (e.g. chronic systemic inflammation and/or intestinal inflammation) in subjects having type 2 diabetes, pre-diabetes and/or symptoms thereof. In some examples, the inflammation may be intestinal and/or pancreatic inflammation. In addition, the inventors surprisingly observed that in patients with type 2 diabetes, changes in lipid profiles (triglyceride concentrations, total cholesterol and HDL-cholesterol) and other parameters of metabolic syndrome were more favourable with AB001 than with placebo. Advantageously, the composition described herein may therefore be used to treat and/or prevent symptoms of metabolic syndrome in subjects having type 2 diabetes and/or pre-diabetes. See Pfützner et al, Fasting Intact Proinsulin Is a Highly Specific Predictor of Insulin Resistance in Type 2 Diabetes, Diabetes Care 27:682-687, 2004, incorporated herein by reference, for further information.

Symptoms of metabolic syndrome may be observed in patients having type 2 diabetes and/or pre-diabetes. As used herein, “metabolic syndrome” refers to the combination of insulin resistance, dyslipidemia and hypertension. Symptoms of metabolic syndrome include, but are not limited to hypertension, hypertriglyceridemia, hyperglycemia, hyperuricemia and other metabolic disorders. The invention can be used to improve symptoms of metabolic syndrome. As described elsewhere herein, in patients with type 2 diabetes, changes in lipid profiles (triglyceride concentrations, total cholesterol and HDL-cholesterol) and other parameters of metabolic syndrome were more favourable with AB001 than with placebo. Advantageously, the composition described herein may therefore be used to treat and/or prevent symptoms of metabolic syndrome in subjects having type 2 diabetes and/or pre-diabetes.

Accordingly, in some examples, administration of the composition to the subject (e.g. a subject having type 2 diabetes, pre-diabetes and/or a symptom thereof):
(a) may reduce the level of one or more of HbA1c or glucose;
(b) may reduce the level of fasting glucose;
(c) may reduce the level of insulin;
(d) may reduce the HOMA-IR score for insulin resistance;
(e) may reduce intact pro-insulin;
(f) may reduce hsCRP in the blood; and/or
(g) may increases the level of adiponectin in the blood.

It is suggested that a result of AB001 supplementation is a lower absorption of glucose through the intestine tract. Accordingly, in some examples, administration of the composition to the subject (e.g. a subject having type 2 diabetes, pre-diabetes and/or a symptom thereof) reduces absorption of glucose into the blood of the subject. A person of skill in the art would be able to determine a reduction in absorption of glucose into the blood in the presence of the composition provided herein (compared to when the composition is not used) using methods known in the art (e.g. by measuring blood glucose in the presence or absence of the composition described herein, using routine methods known in the art). Reduction of glucose in the blood and in the interstitial fluid by the composition would be considered by a person of skill to be an indicator of major improvement of macrovascular risk and general health.

AB001 has no active hypoglycemic action and does not affect normal glucose levels in healthy people or patients with diabetes. This has been observed in a variety of clinical applications and individual patient cases. In clinical studies, AB001 was not associated with an increased risk of hypoglycemia and the suggested mode of action would also not be in line with such findings.

Accordingly, in some examples, administration of the composition to the subject (e.g. a subject having type 2 diabetes, pre-diabetes and/or a symptom thereof):
(a) reduces the level of one or more of HbA1c, glucose (e.g. fasting glucose), insulin, intact pro-insulin or hsCRP in the blood;
(b) reduces the HOMA-IR score; and/or
(c) increases the level of adiponectin in the blood.

In some examples, administration of the composition to the subject (e.g. a subject having type 2 diabetes, pre-diabetes and/or a symptom thereof):
(a) reduces the level of two or more of HbA1c, glucose (e.g. fasting glucose), insulin, intact pro-insulin or hsCRP in the blood;
(b) reduces the HOMA-IR score; and/or
(c) increases the level of adiponectin in the blood.

In some examples, administration of the composition to the subject (e.g. a subject having type 2 diabetes, pre-diabetes and/or a symptom thereof):
(a) reduces the level of three or more of HbA1c, glucose (e.g. fasting glucose), insulin, intact pro-insulin or hsCRP in the blood;
(b) reduces the HOMA-IR score; and/or
(c) increases the level of adiponectin in the blood.

In some examples, administration of the composition to the subject (e.g. a subject having type 2 diabetes, pre-diabetes and/or a symptom thereof) reduces the level of one or more of (e.g. two or more of, or three or more of) HbA1c, glucose (e.g. fasting glucose), insulin, intact pro-insulin or hsCRP in the blood. In some examples, administration of the composition to the subject (e.g. a subject having type 2 diabetes, pre-diabetes and/or a symptom thereof) reduces the HOMA-IR score.

Typically, the level of HbA1c, glucose (e.g. fasting glucose), insulin, intact pro-insulin or hsCRP in the blood and the HOMA-IR score of a subject is determined from a blood sample obtained from the subject, in particular a whole blood sample or a sample drawn from whole blood (e.g. serum and/or plasma). A person of skill in the art would readily be able to identify a suitable sample type depending on the parameter to be determined.

The terms “decrease”, “decreased”, “reduce” “reduced”, “reduction” or “down-regulated”, “lower” are all used herein generally to mean a decrease (e.g. by an amount (for instance, a statistically significant amount)). For the avoidance of doubt, “reduced”, “reduce”, “reduction”, “decreased” or “decrease” in the context of the present invention mean a decrease as compared to a reference level/control.

A reference level/control in the context of the present invention may be the level prior to administration of the composition described herein.

In some examples, administration of the composition to the subject (e.g. a subject having type 2 diabetes, pre-diabetes and/or a symptom thereof) reduces the level of HbA1c in the blood. In other words, administration of the composition to the subject (e.g. a subject having type 2 diabetes, pre-diabetes and/or a symptom thereof) reduces the level of HbA1c in the blood as compared to the level of HbA1c in the blood before administration of the composition. HbA1c levels may be determined using routine methods known in the art, as described in the examples section below, for example the HbA1c test may be used. HbA1c and the HbA1c test is discussed in more detail elsewhere herein. HbA1c levels may be used to identify impaired glucose regulation in a subject and thus, as described in the examples section below, may be used as a biomarker of glycemic control.

In some examples, administration of the composition to the subject (e.g. a subject having type 2 diabetes, pre-diabetes and/or a symptom thereof) reduces the level of glucose (e.g. fasting glucose) in the blood. In other words, administration of the composition to the subject (e.g. a subject having type 2 diabetes, pre-diabetes and/or a symptom thereof) reduces the level of glucose in the blood (e.g. after fasting) as compared to the level of glucose in the blood before administration of the composition (e.g. the level of glucose in the blood before administration of the composition after fasting). Blood glucose may be determined using routine methods known in the art (as described in the examples section below), for example, fasting blood glucose may be determined using the FPG test discussed in more detail elsewhere herein. Fasting glucose level may be used as a biomarker of glycemic control, as discussed in the examples section below.

As used herein, “fasting blood glucose” refers to the level of glucose in the blood after a fast. Typically, in the context of the present invention, a fast will last between 8-12 hours, e.g. between 8-10 hours, and in this period of time no food or drink is consumed by the subject (in some instances, a small amount of water may be consumed). Accordingly, “fasting blood glucose” as used herein typically refers to the level of glucose in the blood after a fast for at least 8 hours, for example after fasting for 8 to 12 hours.

In some examples, administration of the composition to the subject (e.g. a subject having type 2 diabetes, pre-diabetes and/or a symptom thereof) reduces the level of insulin in the blood. In other words, administration of the composition to the subject (e.g. a subject having type 2 diabetes, pre-diabetes and/or a symptom thereof) reduces the level of insulin in the blood as compared to the level of insulin in the blood before administration of the composition. Insulin is a peptide hormone secreted in the body by beta cells of islets of Langerhans of the pancreas and regulates blood glucose levels. Insulin acts by directly binding to its receptors on the plasma membranes of the cells. These receptors are present on all the cells, but their density depends on the type of cells, with the maximum density being on the hepatic cells and adipocytes. The insulin receptor is a heterotetrameric glycoprotein consisting of two subunits, the alpha and the beta subunits. The extracellular alpha subunits have insulin binding sites. The beta subunits, which are transmembranous, have tyrosine kinase activity. When insulin binds to the alpha subunits, it activates the tyrosine kinase activity in the beta subunit, which causes the translocation of glucose transporters from the cytoplasm to the cell's surface. These glucose transporters allow the influx of glucose from the blood into the cell, thus reducing the blood glucose levels.

Insulin causes the following effects in the cells:
hepatic cells: promotes glycogenesis, inhibits gluconeogenesis;
adipocytes: promotes lipogenesis, inhibits lipolysis;
muscle cells: promotes glycogenesis and protein synthesis, inhibits protein catabolism;
pancreatic beta cells: inhibits glucagon release; and
brain cells: involved in appetite regulation.

Conditions such as diabetes tend to increase the level of insulin in the blood. A person of skill in the art would readily be able to determine insulin levels in the blood using routine methods known in the art (as described in the examples section below). As would be known to a person of skill in the art, blood insulin levels may be used as a biomarker of insulin resistance as discussed in the examples section below.

In some examples, administration of the composition to the subject (e.g. a subject having type 2 diabetes, pre-diabetes and/or a symptom thereof) reduces HOMA-IR score. In other words, administration of the composition to the subject (e.g. a subject having type 2 diabetes, pre-diabetes and/or a symptom thereof) reduces the HOMA-IR score as compared to the HOMA-IR before administration of the composition. “HOMA-IR” refers to the homeostasis model assessment-estimated insulin resistance. The homeostasis model assessment-estimated insulin resistance (HOMA-IR), developed by Matthews et al, has been widely used for the estimation of insulin resistance in research. Compared with the “gold” standard euglycemic clamp method for quantifying insulin resistance, quantification using HOMA-IR is more convenient. It is calculated multiplying fasting plasma insulin (FPI) by fasting plasma glucose (FPG), then dividing by the constant 22.5, i.e. HOMA-IR=(FPI×FPG)/22.5. Low HOMA-IR values indicate high insulin sensitivity, whereas high HOMA-IR values indicate low insulin sensitivity (insulin resistance). A person of skill in the art would readily be able to determine fasting plasma insulin (FPI) and fasting plasma glucose (FPG) levels using routine methods known in the art. Accordingly, a person of skill in the art would readily be able to determine the HOMA-IR score for an individual using routine methods known in the art.

In some examples, administration of the composition to the subject (e.g. a subject having type 2 diabetes, pre-diabetes and/or a symptom thereof) reduces the level of intact pro-insulin in the blood. In other words, administration of the composition to the subject (e.g. a subject having type 2 diabetes, pre-diabetes and/or a symptom thereof) reduces the level of intact pro-insulin in the blood as compared to the level of intact proinsulin in the blood before administration of the composition. Proinsulin is produced in the pancreatic ß-cells and is normally further processed to insulin and C-peptide. It is only seen in low concentrations in the blood (e.g. plasma) of healthy subjects. An increase in the insulin demand, as provided by insulin resistance in later stages of type 2 diabetes mellitus, can result in increased expression of proinsulin into the blood. Intact proinsulin is rapidly degraded. In clinical practice, fasting morning intact proinsulin can be used as highly specific indicator of clinically relevant insulin resistance, to serve as the basis for the selection of an insulin resistance therapy, and to monitor the therapeutic effect on ß-cell dysfunction. A person of skill in the art would readily be able to determine intact proinsulin levels in the blood using routine methods known in the art (as described in the examples section below). As would be known to a person of skill in the art, intact proinsulin levels may be used as a biomarker for β-cell dysfunction and/or insulin resistance.

In some examples, administration of the composition to the subject (e.g. a subject having type 2 diabetes, pre-diabetes and/or a symptom thereof) reduces the level of hsCRP in the blood. In other words, administration of the composition to the subject (e.g. a subject having type 2 diabetes, pre-diabetes and/or a symptom thereof) reduces the level of hsCRP in the blood as compared to the level of hsCRP in the blood before administration of the composition. C-reactive protein (CRP) is a protein in the blood that increases when inflammation is present CRP has been used for many years as an indicator of infection and inflammation associated with disease. The hs-CRP test accurately measures low levels of CRP to identify low but persistent levels of inflammation. Accordingly, as would be known to a person of skill in the art, hsCRP levels may be used as a biomarker of inflammation, in particular chronic systemic inflammation. A person of skill in the art would readily be able to determine hs-CRP levels in the blood using routine methods known in the art (as described in the examples section below).

In some examples, administration of the composition to the subject (e.g. a subject having type 2 diabetes, pre-diabetes and/or a symptom thereof) increases the level of adiponectin in the blood. In other words, administration of the composition to the subject (e.g. a subject having type 2 diabetes, pre-diabetes and/or a symptom thereof) increases the level of adiponectin in the blood as compared to the level of adiponectin in the blood before administration of the composition. Adiponectin is the most abundant peptide hormone secreted by adipocytes. Adiponectin is a hormone your adipose (fat) tissue releases that helps with insulin sensitivity and inflammation. Low levels of adiponectin are associated with several conditions, including Type 2 diabetes and atherosclerosis. Adiponectin may be used as a biomarker of inflammation, in particular chronic systemic inflammation. A person of skill in the art would readily be able to determine adiponectin levels in the blood using routine methods known in the art (as described in the examples section below).

The terms “increased”, “increase” or “up-regulated”, “higher” are all used herein to generally mean an increase (e.g. by an amount (for instance, a statically significant amount). For the avoidance of doubt, the terms “increased” or “increase” in the context of the present invention mean an increase as compared to a reference level/control.

A reference level/control in the context of the present invention may be the level prior to administration of the composition described herein.

In some examples, administration of the composition to the subject (e.g. a subject having type 2 diabetes, pre-diabetes and/or a symptom thereof) controls blood glucose (e.g. whole blood glucose, plasma glucose and/or serum glucose) and/or interstitial glucose in the subject.

In some examples, administration of the composition to the subject (e.g. a subject having type 2 diabetes, pre-diabetes and/or a symptom thereof) controls blood glucose (e.g. whole blood glucose, plasma glucose and/or serum glucose) in the subject.

In some examples, administration of the composition to the subject (e.g. a subject having type 2 diabetes, pre-diabetes and/or a symptom thereof) controls interstitial glucose in the subject.

Accordingly, in a particular example, administration of the composition to the subject (e.g. a subject having type 2 diabetes, pre-diabetes and/or a symptom thereof) stabilises the level of blood glucose and/or the level of interstitial glucose in the subject. In a particular example, administration of the composition to the subject (e.g. a subject having type 2 diabetes, pre-diabetes and/or a symptom thereof) stabilises the level of blood glucose in the subject. In another example, administration of the composition to the subject (e.g. a subject having type 2 diabetes, pre-diabetes and/or a symptom thereof) stabilises the level of interstitial glucose in the subject.

As discussed elsewhere herein, it is suggested that AB001 has four glucose-controlling actions. For instance, it is suggested that the Bacilli strains of AB001 and their excreted enzymes act as additional glucose-digesters (in other words, the Bacilli strains of AB001 and their excreted enzymes act in addition to the microbiome (e.g. the gut microbiome) of a subject that was present before consumption of AB001) to further reduce resorption of glucose to the blood. Accordingly, in some examples, administration of the composition to the subject (e.g. a subject having type 2 diabetes, pre-diabetes and/or a symptom thereof) degrades glucose. In some examples, administration of the composition to the subject degrades glucose in the gut of the subject. In some examples, administration of the composition to the subject degrades glucose in the intestine (in particular, the small intestine) of the subject. More specifically, administration of the composition for use according to the present invention to the subject degrades glucose in the duodenum of the subject. Glucose degradation and glucose degrading compositions for use according to the present invention are described elsewhere herein. The text provided elsewhere herein in relation to “glucose degradation” and “glucose degrading compositions” applies equally here.

As discussed elsewhere herein, glucose may be metabolized (i.e. glucose may be degraded via metabolism). Accordingly, in some examples, administration of the composition to the subject (e.g. a subject having type 2 diabetes, pre-diabetes and/or a symptom thereof) metabolises glucose. In some examples, administration of the composition to the subject metabolises glucose in the gut of the subject. “Metabolism” is defined elsewhere herein and applies equally here. In some examples, administration of the composition for use according to the present invention to the subject metabolises glucose in the intestine of the subject. In one example, administration of the composition for use according to the present invention to the subject metabolises glucose in the small intestine of the subject. More specifically, administration of the composition for use according to the present invention to the subject metabolises glucose in the duodenum of the subject.

It is suggested that a result of AB001 supplementation is a lower absorption of glucose through the intestine tract. Accordingly, in some examples, administration of the composition to the subject (e.g. a subject having type 2 diabetes, pre-diabetes and/or a symptom thereof) reduces absorption of glucose into the blood of the subject. A person of skill in the art would be able to determine a reduction in absorption of glucose into the blood in the presence of the composition provided herein (compared to when the composition is not used) using methods known in the art (e.g. by measuring blood glucose in the presence or absence of the composition described herein, using routine methods known in the art).

In some examples, administration of the composition to the subject (e.g. a subject having type 2 diabetes, pre-diabetes and/or a symptom thereof) reduces absorption of glucose into the interstitial fluid of the subject. A person of skill in the art would be able to determine a reduction in absorption of glucose into the interstitial fluid in the presence of the composition provided herein (compared to when the composition is not used) using methods known in the art (e.g. by measuring interstitial glucose in the presence or absence of the composition described herein, using routine methods known in the art).

In some examples, administration of the composition to the subject (e.g. a subject having type 2 diabetes, pre-diabetes and/or a symptom thereof) reduces blood glucose and/or interstitial glucose concentration in the subject. A person of skill in the art would be able to determine a reduction in blood glucose concentration and/or interstitial glucose concentration in the presence of the composition provided herein (compared to when the composition is not used) using methods known in the art.

In some examples, administration of the composition to the subject (e.g. a subject having type 2 diabetes, pre-diabetes and/or a symptom thereof) reduces blood glucose concentration in the subject. A person of skill in the art would be able to determine a reduction in blood glucose concentration in the presence of the composition provided herein (compared to when the composition is not used) using methods known in the art.

In some examples, administration of the composition to the subject (e.g. a subject having type 2 diabetes, pre-diabetes and/or a symptom thereof) reduces interstitial glucose concentration in the subject. A person of skill in the art would be able to determine a reduction in interstitial glucose concentration in the presence of the composition provided herein (compared to when the composition is not used) using methods known in the art.

The compositions provided herein are effective when they are administered to a subject for an appropriate length of time, at an appropriate frequency. An appropriate length of time and frequency may be readily identified by a person of skill in the art or a corresponding physician.

In some examples, the composition (e.g. an effective amount of the composition described herein) is ingested. In some examples, the composition (e.g. an effective amount of the composition described herein) is ingested daily for an appropriate length of time. In other words, the composition for use according to the present invention may be for daily administration.

In some examples, the composition (e.g. an effective amount of the composition described herein) is ingested for at least 1 day. In some examples, the composition (e.g. an effective amount of the composition described herein) is ingested daily for at least 2 days, at least 3 days, at least 4 days, at least 5 days, at least 6 days or at least 7 days.

In some examples, the composition (e.g. an effective amount of the composition described herein) is ingested daily for at least one week. In some examples, the composition (e.g. an effective amount of the composition described herein) is ingested daily for at least two weeks.

In some examples, the composition (e.g. an effective amount of the composition described herein) is ingested daily for at least four weeks. In some examples, the composition (e.g. an effective amount of the composition described herein) is ingested daily for at least six weeks. In some examples, the composition (e.g. an effective amount of the composition described herein) is ingested daily for at least six months.

For instance, one dose (e.g. 2 capsules or tablets of the composition described herein) may be taken daily for at least 2 days, at least 3 days, at least 4 days, at least 5 days, at least 6 days or at least 7 days. For instance, one dose (e.g. 2 capsules or tablets of the composition described herein) may be taken daily for 2 days, 3 days, 4 days, 5 days, 6 days or 7 days.

For instance, one dose (e.g. 2 capsules or tablets of the composition described herein) may be taken daily for at least one week, at least 2 weeks, at least 3 weeks, at least 4 weeks, at least 5 weeks or at least 6 weeks. For instance, one dose (e.g. 2 capsules or tablets of the composition described herein) may be taken daily for one week, two weeks, 3 weeks, 4 weeks, 5 weeks or 6 weeks.

“Ingestion” as used herein, refers to the taking in (e.g. consumption) of a substance by an organism (e.g. a subject, preferably a human). In the context of the present invention, ingestion is preferably via the mouth where the subject is human.

As would be clear to a person of skill in the art, the compositions for use according to the present invention described herein may be used in combination with one or more other treatments (e.g. medications) that are used to prevent and/or treat type 2 diabetes, pre-diabetes and/or a symptom thereof. Accordingly, the compositions for use according to the present invention described herein may be used to supplement the one or more other treatments (e.g. medications) used to prevent and/or treat type 2 diabetes, pre-diabetes and/or a symptom thereof.

In one example, the compositions for use according to the present invention described herein may be administered to (e.g. ingested by) a subject that is undergoing treatment with one or more treatments (e.g. medications) that are used to prevent and/or treat type 2 diabetes, pre-diabetes and/or a symptom thereof. Treatments (e.g. medications) that are used to prevent and/or treat type 2 diabetes, pre-diabetes and/or a symptom thereof are well known in the art. Non-limiting examples of treatments (e.g. medications) that are used to prevent and/or treat type 2 diabetes, pre-diabetes and/or a symptom thereof are described in the examples section below.

The composition for use according to the present invention (e.g. the composition for use in treating and/or preventing type 2 diabetes, pre-diabetes, and/or a symptom thereof in a subject, and/or in a method of treating and/or preventing type 2 diabetes, pre-diabetes, and/or a symptom thereof in a subject) may be orally ingested in the form of an aqueous solution, a tablet, a capsule, a granule, or the like.

The compositions described herein are for administration to a subject (preferably a human) in an effective amount (in an effective dose). An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the subject. For example, a suitable dosage of the composition of the invention for a given subject can be determined by a physician (or the person administering the composition), taking into consideration various factors known to modify the action of the composition of the invention for example body weight, sex, diet, time and route of administration, other medications and other relevant clinical factors. The dosages and schedules may be varied according to the particular condition, disorder or symptom the overall condition of the subject. Effective dosages may be determined by either in vitro or in vivo methods.

The compositions described herein are advantageously presented in dosage units. For example, the composition may be presented in the form of a capsule or a tablet. Other suitable dosage units are described elsewhere herein.

An “effective amount” may comprise administration of one or more dosage units. For example, an effective amount may be achieved by administration of one or two capsules or tablets. When the effective amount comprises a plurality of dosage units, the dosage units may be administered together, or they may be taken at spaced intervals during the day.

Alternative appropriate effective amounts and dosage forms will be readily identifiable by a person of skill in the art, based on the examples below, using routine experimentation.

As used herein, the terms “treating” and “treatment” refer to the administration of a composition to a subject (e.g., a symptomatic subject afflicted with an adverse condition, disorder, illness or disease (e.g. type 2 diabetes, pre-diabetes and/or a symptom thereof) so as to affect a reduction in severity and/or frequency of a symptom, eliminate a symptom and/or its underlying cause, and/or facilitate improvement or remediation of damage, and/or preventing an adverse condition, disorder, illness or disease in an asymptomatic subject who is susceptible to a particular adverse condition, disorder, illness or disease, or who is suspected of developing or at risk of developing the condition, disorder, illness or disease.

By way of example, as described elsewhere herein, treatment with the composition described herein may advantageously:
(a) reduce the level of one or more of HbA1c, glucose (e.g. fasting glucose), insulin, intact pro-insulin or hsCRP in the blood;
(b) reduce the HOMA-IR score; and/or
(c) increase the level of adiponectin in the blood.

Treatment with the composition described herein may also advantageously control blood glucose, reduce absorption of glucose into the blood of the subject and/or reduce blood glucose concentration in the subject.

The term “prevention” as used herein means the avoidance of the occurrence or re-occurrence a symptom and/or its underlying cause, damage, an adverse condition, disorder, illness and/or disease. For instance, “prevention” in the context of the present invention may be avoiding the appearance of one or more of the symptoms related to type 2 diabetes and/or pre-diabetes.

As discussed elsewhere herein, the subject may be any suitable subject, for example, the subject may be human. Preferably, the subject is human. The subject may be a human has been having type 2 diabetes, pre-diabetes and/or a symptom thereof. A clinician would readily be able to diagnose type 2 diabetes, pre-diabetes and/or a symptom thereof using routine methods known in the art, for example using the tests and reference values discussed elsewhere herein. The subject may be a human diagnosed with type 2 diabetes, pre-diabetes and/or a symptom thereof that is intending to consume or has consumed glucose.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Development of a Composition Described Herein

The inventors have previously developed a food grade quality of fermented rice bran for evaluation purposes. A safety study was initiated applying a case study design. Around 1,400 people have taken the product on a regular long-term basis. No side effects/adverse reactions have been reported.

First indication of effects on glucose could be concluded from the safety trial, case study design, using oral administration of fermented rice bran including Bacilli strains (without L-cysteine and without dextrin), where Type-2 diabetics reported extended euglycemia (self test), and increased levels of HDL, lower levels of triglycerides and lowered HbA1c levels were reported at regular medical check-ups every three to six months. By physicians' ordination some of these individuals came off one daily dose of sensitiser and some reduced the dose or came off statins completely. Some Type-2 diabetics showed HbA1c and other blood parameters typical for prediabetics after three to six months. (Data not shown). This safety trial indicates that a composition comprising fermented rice bran and Bacilli strains (without L-cysteine and without dextrin) is beneficial to patients with type 2 diabetes.

These findings led to the AB001 formulation, fermented rice bran including Bacilli strains to ˜70%, that has been evaluated in a randomised, placebo-controlled clinical trial confirming positive effects on euglycemia, LDL-HDL ratio, triglycerides, HbA1c and more. The results of this clinical trial are shown in example 1.

Prediabetes is a pre-stage of Type-2 diabetes and therefore, from scientific and clinical perspectives, the data discussed herein indicates that fermented rice bran/Bacilli strains may be an efficient and effective treatment for prediabetes. The composition may be used to avoid/prevent the medical condition developing into Type-2 diabetes).

The composition described herein (AB001) is typically composed of naturally fermented rice bran, Bacillus subtilis and Bacillus coagulans, L-cysteine, and dextrin. It may also include magnesium stearate and calcium and potassium phosphates. The supplement comes in acid resistant capsules, HPMC, which are dissolved once reaching the duodenum. The cultures are released and settle in the upper part of the intestinal tract where they stay for about one day before being eliminated from the body through the feces. The bacterial strains included in the composition were selected to preferably and effectively metabolize glucose.

The bacterial composition of AB001 was investigated using Sanger DNA sequencing. It was identified that the dominant bacterial strains found in the compositions used were Bacillus subtilis spp and Bacillus coagulans spp. Other genus/species identified were Bacillus amyloliquefaciens, Bacillus velezensis, Bacillus sp MT 03, Bacillus atrophaeus, and Pediococcus pentosaceus.

Dextrin from three different suppliers was tested and confirmed to work in the compositions of the invention (data not shown). The preferred dextrin that was used herein was hydrolysed corn dextrin (e.g. Vitargo). L-cysteine from three different suppliers was also tested and confirmed to work (data not shown). The preferred L-cysteine that was used herein was of vegetable origin.

The composition used in example 1 comprised, per dose (of 2 capsules): fermented rice bran (552 mg), L-cysteine (150 mg), microcrystalline cellulose (40 mg), magnesium stearate (4.8 mg), dextrin (4 mg), silicon dioxides (4 mg) and at least 1×10₅ cfu of bacteria of the Bacillus genus. Optionally, Chromium (e.g. Chromium Chloride) may additionally be added for regulatory purposes. Optionally, at least about 20 mcg of chromium per dose may be added for regulatory purposes. For example, about 80 mcg to about 100 mcg of chromium chloride per dose may be added for regulatory purposes.

### Example 1—Impact of a Bacterial-Based Nutritional Supplement (AB001) on Biomarkers of Glycemic Control and Metabolic Syndrome in Patients with Type 2 Diabetes Mellitus

### Summary

Background: AB001 is a nutritional supplement based on a co-existing microbial consortium of bacterial strains. The purpose of this study was to investigate potential beneficial effects of the supplement vs. placebo on glycemic control and on biomarkers of inflammation, insulin resistance and β-cell dysfunction.

Methods: A total of 40 Patients with type 2 diabetes (31 male, 9 female, age: 65.5±8.0 yrs, BMI: 33.6±5.5 kg/m², HbA1c: 7.2±0.9%) were included into the study. They were randomized to receive either AB001 or placebo once daily for 6 weeks. Specifically, two capsules of the composition (e.g. one dose of the composition) were taken per day by participants in the treatment arm of the trial. Observation parameters were time in normoglycemia as assessed by continuous glucose monitoring, and biomarkers of glycemic control, ß-cell function, insulin resistance and chronic systemic inflammation.

Results: Time in range was stable with AB001 between weeks 0-2 vs. weeks 4-6, while it slightly impaired with placebo (−4%). At endpoint, there were significant improvements vs. baseline with AB001, but not with placebo for biomarkers of glycemic control (AB001 vs. Placebo, HbA1c: −0.3% vs. 0.1%, fasting glucose: −14% vs. +8%, both p<0.05), insulin resistance (insulin: −17% vs. +14%, HOMA-IR: −26% vs. +21%, both p<0.05), ß-cell dysfunction (intact proinsulin: −40% vs. −2%, p<0.05) and chronic systemic inflammation (adiponectin: +8% vs. −8%; p<0.05, hsCRP: −31% vs. +27%, n.s.). In addition, observed changes in the lipid profiles and other parameters of metabolic syndrome in TIID patients were more favourable with AB001 than with placebo. There were no differences between the groups with respect to number and type of adverse events.

Conclusion: The results observed with AB001 were comprehensively indicative for improvements in the cardiometabolic situation. AB001 is a valuable supplement to any existing treatment combination in patients with type 2 diabetes.

### Introduction

As described elsewhere herein, de Faire Medical AB (Stockholm, Sweden, www.defairemedical.com) has developed AB001, a nutritional supplement based on Fermented Rice Bran with Bacillus subtilis and Bacillus coagulans, which constitute the prebiotic and probiotic properties of the product. In part, the product action is driven by excreting bioactive substances, such as enzymes, that break down complex organic molecules into nutrients, reactants and energy. For example, the bacterial strains included into the product, e.g. Bacillus subtilis and B. coagulans, combined with Dextrin and L-Cysteine, preferably metabolize glucose and other more complex carbohydrate molecules. A more detailed mechanism of action is described elsewhere herein. AB001 has no active hypoglycemic action and does not affect normal glucose levels in healthy people or patients with diabetes. The product complies the European regulatory framework as a nutritional supplement. In this double-blind study, AB001 or placebo was given as a nutritional supplement in addition to standard of care treatment interventions for six weeks in patients with type 2 diabetes mellitus in later disease stages.

### Patients and Methods

This prospective double-blind placebo-controlled monocentric parallel study was conducted in accordance with international and local ethical and scientific standards. The protocol was approved by the responsible ethical review board (Landesärztekammer Rheinland-Pfalz, Mainz, Germany), and reported to the responsible national authority (Bundesamt für Verbraucherschutz und Lebenmittelsicherheit). The study was registered at the German Registry for Clinical Studies (DRKS No. DRKS00023745).

Primary objective was the impact of AB001 on glycemic control (time in normoglycemia) as compared to placebo and as assessed by continuous glucose measurement by means of the Freestyle Libre 2 device (Abbott Diagnostics, Wiesbaden-Delkenheim, Germany) after 2, 4, and 6 weeks of treatment. Secondary objectives were the effects of the dietary supplement vs. placebo on HbA1c, lipids, uric acid, insulin sensitivity (as assessed by HOMA-score [16]), ß-cell dysfunction (insulin and intact proinsulin [17, 18]), chronic systemic inflammation and cardiovascular risk (total adiponectin and hsCRP, [19, 20]), and the tolerability of the nutritional supplement (type and nature of (serious) adverse events).

To be eligible for the study, patients had to be adult and diagnosed with 2 diabetes (HbA1c between 6.0% and 9.9%, while treated with any kind or combination of standard of care treatments). They were not allowed to suffer from type 1 diabetes, any acute or chronic gastrointestinal disease, anemia, acute hyperthyreosis, known allergy against probiotic nutritional supplements, or any major fatal disease.

Prior to participation the participants signed written informed consent. Thereafter, blood was drawn for the safety analysis and to identify potential exclusion criteria. The randomization into the two study arms and blood draws for determination of efficacy parameters took place at the baseline visit (V1). The entire observation period was six weeks. A subcutaneous sensor for continuous glucose monitoring (CGM, FreeStyle Libre, Abbott) was provided for the next two weeks. Three follow-up visits (V2, V3, V4) were scheduled to happen in 2-week intervals to exchange the CGM sensor and to draw blood for the efficacy analysis. Conclusion of visit 4 terminated the participation of the individual patient in the study.

All safety and efficacy parameters were measured at a central laboratory. Immunoassays were used for determination of insulin, intact proinsulin and total adiponectin (TecoMedical, Sissach, CH). Lipids, hsCRP, glucose, and HbA1c were determined with a standard laboratory analyzer (Cobas c513, Roche Diagnostics, Basel, CH). At each treatment visit, the patients were asked for occurrence of any (serious) adverse events.

The data was evaluated using methods of standard exploratory and descriptive analyses to gain an understanding of the qualitative and quantitative nature of the collected data. For quantitative variables arithmetic means, medians, standard deviations, and minimum and maximum values were determined. Appropriate parametric and non-parametric statistical tests were used to compare the collected results. For normally distributed data sets, students t-test was used to compare the changes from baseline to endpoint, and to compare the changes from baseline between the groups. A p-value <0.05 was considered to be statistically significant.

The composition used comprised, per dose (of 2 capsules): fermented rice bran (552 mg), L-cysteine (150 mg), microcrystalline cellulose (40 mg), magnesium stearate (4.8 mg), dextrin (4 mg), silicon dioxides (4 mg), and at least 1×10⁵ cfu of bacteria of the Bacillus genus. Optionally, Chromium (e.g. Chromium Chloride) may additionally be added for regulatory purposes. Optionally, at least about 20 mcg of chromium per dose may be added for regulatory purposes. For example, about 80 mcg to about 100 mcg of chromium chloride per dose may be added for regulatory purposes.

Two capsules of the composition (e.g. one dose of the composition) were taken per day by the participants in the treatment arm of the trial.

### Results

A total of 46 patients were screened of whom 40 meeting the inclusion/exclusion criteria could be enrolled into the study (31 men, 9 women, mean age: 65.4±8.0 yrs (range: 44 yrs.-89 yrs.), BMI: 33.3±5.3 kg/m² (range: 21.5 kg/m²-42.8 kg/m²). HbA1c: 7.2±0.9% (55±10 mmol/mol; range: 6.0%-9.4%/42-79 mmol/mol)). All included patients performed the study per protocol. After randomization, there were slightly more women in the placebo arm. The vast majority of the patients were on combination treatments with multiple anti-diabetic drugs indicating an advanced disease stage. The patient characteristics at baseline for both treatment arms and the nature and distribution of the diabetes treatments are provided in Table 1.

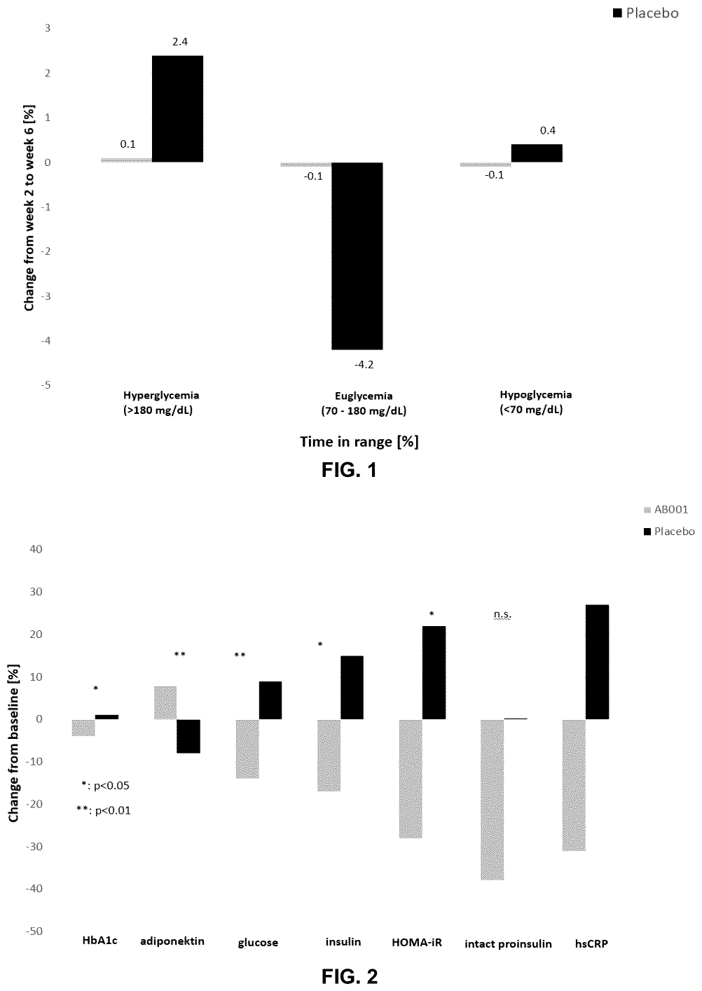

Primary measurement variable was time in normoglycemia as determined by means of the FreeStyle Libre analysis software between weeks 0 to 2 and week 4 to 6. It must be noted that there was no data available from the time before the patients started to take the study supplements. Therefore, a true baseline to understand the immediate impact of AB001 supplementation on time in normoglycemia for the comparison is missing. The comparison between the first and the last sensor analysis in this trial is provided in FIG. 1.

It can be seen that patients taking AB001 had stable glycemic control, while a slight but non-significant impairment could be observed in the placebo group.

The differences that were seen in the majority of the clinical examination parameters (body weight, blood pressure, waist circumference) were numerically more favorable for AB001 but not significant. Only an observed reduction in hip circumference was significantly more pronounced with AB001 (from 121±11 cm to 116±11 cm, p<0.005) as compared to placebo (116±11 cm vs. 116±10 cm, n.s.).

To explore the impact of AB001 on biomarkers of glycemic control and the underlying diabetes deteriorations (insulin resistance, ß-cell dysfunction and chronic systemic inflammation) a panel of biochemical parameters were measured at baseline and endpoint. The results are provided in Table 2 and the percent changes from baseline in both groups are provided in FIG. 2.

Hemoglobin A1c as well as fasting glucose improved significantly with AB001, while no change (HbA1c) or an impairment (fasting glucose) was seen with placebo.

Intact proinsulin, insulin and the HOMA-IR score were determined as indicators for insulin resistance and ß-cell dysfunction [16-18]. All of them improved significantly with AB001, while they further deteriorated with placebo. All changes from baseline with AB001 as well as the differences between the groups reached the level of statistical significance. These findings are indicative for a pronounced positive impact of AB001 on insulin resistance in this trial.

Biomarkers indicative for chronic systemic inflammation determined in this study were total adiponectin and hsCRP [19, 20]. Adiponectin increased significantly with AB001 and decreased with placebo. These observations were also highly significant for changes between the groups (p<0.001). AB001 lead to a numerical reduction in hsCRP by more than 30%, while it increased by more than 20% with placebo. However, due to a high variability observed, the changes between the groups did not reach the level of statistical significance.

With respect to other laboratory biomarkers of metabolic syndrome (triglycerides, cholesterol, and uric acid) an almost similar picture could be observed (see Table 2). There was a statistically significant improvement in the triglyceride concentrations with AB001 but not with placebo. There were strong trends in favor of AB001 for the total cholesterol and HDL-cholesterol levels. Observed impairments for LDL-cholesterol and uric acid were less pronounced with AB001 than with placebo. These findings are in line with the overall positive laboratory picture observed with AB001 in comparison to placebo.

The supplements were well tolerated, and there were no serious adverse events reported in this trial. A total of 33 adverse events were reported, of which 18 occurred with placebo and 15 occurred with AB001. Symptoms associated with problems occurring in the gastro-intestinal tract were seen in 6 cases with placebo and only one case with AB001. Hypoglycemia was reported 6 times from 2 patients with AB001 and 4 times from 4 patients with placebo. From the reported adverse events it can be concluded that the nutritional supplement had no special side effects in this trial. In addition, there were no clinically relevant changes during the study in any other value of the safety biochemistry panels taken before and after the study.

### Discussion

Diabetes mellitus is a chronic systemic disease associated with complications in most of the body organs. It is a pandemic, which affects ˜9% of the population in the world [21].

Changes in pancreatic beta-cell function and insulin resistance lead to relative insulin deficiency and impaired cellular response to insulin. A significant symptom of the disease is hyperglycemia, which can cause several severe secondary complications [22, 23]. Anti-diabetic drugs are commonly used in accordance with national and international treatment guidelines [24, 25] and the most commonly used drug, metformin, can have limited effect and can cause adverse effects on the composition of the bacterial microfauna in the intestine tract. Recently, the gut microbiome was found to be altered in diabetic patients, e.g. with increased ratios of bacteriocides to clostridium species in the gut, and increased numbers of various opportunistic pathogens [26-29]. Circulating gram-positive gut bacteria were detected in blood samples of diabetic patients [28]. Changes in gut microbiome may lead to metabolic endotoxemia through the release of lipopolysaccharides, consequently stimulating inflammation and insulin resistance [30]. The epithelial enteroendocrine L cells play a role in producing inflammation, and their number was positively or negatively correlated with the abundance of 25 bacterial taxa in the intestine [31, 32].

In light of these findings, the effect of probiotic supplementation in diabetes and diabetes-related complications gets into the focus of modern research. Animal studies showed positive results, such as reductions in levels of blood glucose, hemoglobin A1c (HbA1c), and insulin resistance [33, 34]. Also, first studies in humans were performed to investigate the clinical effect of probiotics on glycemic control [14, 35, 36]. In these placebo-controlled trials, probiotic fermented milk, probiotic yogurt, bread, and kefir mainly containing different strains of Lactobacillae, and Brewer's yeast were administered for 6 to 12 weeks. In a meta-analysis of these studies, significant improvements were reported for HbA1c, fasting glucose and the HOMA-IR score, while non-significant improvements were seen in insulin and fasting proinsulin levels [37].

The AB001 development is different to these interventions in so far as special bacterial strains have been selected, which preferably metabolize glucose and other carbohydrates, and which should therefore provide an even stronger impact on glucose levels and insulin resistance. Within the limitations of this study protocol, this hypothesis of an even more pronounced effect is confirmed, in particular when employing a laboratory biomarker panel, which describes the diabetes phenotype by indicating the severity of the underlying diabetes deteriorations [38].

A result of AB001 supplementation seems to be a lower absorption of glucose through the intestine tract. A similar effect and corresponding biochemical findings were seen, when drugs belonging to the class of SGLT-1 inhibitors (α-glucosidase inhibitors, e.g. acarbose or voglibiose) are used in routine diabetes treatment. The difference between the two approaches is that SGLT-1 inhibitors actively withhold the glucose molecules in the intestine tract, where they finally get metabolized by bacteria populating the large bowel, with methane gas as the final end product. In consequence, patients treated with SGLT-1 inhibitors were usually complaining about major problems with flatulence and bloated belly [39, 40]. The majority of the patients in both treatment arms were already using insulin in different treatment regimens, indicating a somewhat advanced disease stage. The positive effects of AB001 on biomarkers of the underlying diabetes deteriorations must be quite pronounced that consistently positive significant results were seen for almost all of the investigated indicators.

### CONCLUSIONS

The results observed with AB001 were comprehensively indicative for improvements in the cardiometabolic situation, while the opposite was seen for all parameters with placebo.

Considering the intensive background diabetes treatments taken by the participating patients, these results are a strong indicator that AB001 is a valuable supplement to any existing treatment combination in patients with type 2 diabetes.

### REFERENCES

1. Gentile CL, Weir TL. The gut microbiota at the intersection of diet and human health. Science. 2018; 362:776-780.
2. Lynch SV, Pedersen O. The Human Intestinal Microbiome in Health and Disease. N Engl J Med. 2016; 375:2369-2379.
3. Postler, T. S.; Ghosh, S. Understanding the Holobiont: How Microbial Metabolites Affect Human Health and Shape the Immune System. Cell Metab. 2017, 26:110-130.
4. Bibbò, S.; Ianiro, G.; Giorgio, V.; Scaldaferri, F.; Masucci, L.; Gasbarrini, A.; Cammarota, G. The role of diet on gut microbiota composition. Eur. Rev. Med. Pharmacol. Sci. 2016,20, 4742-4749.
5. Sender, R.; Fuchs, S.; Milo, R. Revised Estimates for the Number of Human and Bacteria Cells in the Body. PLoS Biol. 2016,14, e1002533.
6. Kåhrström, C. T.; Pariente, N.; Weiss, U. Intestinal microbiota in health and disease. Nature 2016, 535:47
7. Macfarlane, S.; Macfarlane, G.T. Regulation of short-chain fatty acid production. Proc. Nutr. Soc. 2003, 62:67-72
8. Rowland, I.; Gibson, G. R.; Heinken, A.; Scott, K. P.; Swann, J.; Thiele, I.; Tuohy, K. M. Gut microbiota functions: Metabolism of nutrients and other food components. Eur. J. Nutr. 2018, 57:1-24
9. Patterson E, Ryan P M, Cryan J F, Dinan T G, Ross R P, Fitzgerald G F, Stanton C. Gut microbiota, obesity and diabetes. Postgrad Med J. 2016; 92-286-300.
10. Ortega M A, Fraile-Martlnez O, Naya I, García-Honduvilla N, Álvarez-Mon M, Buján J, Ásunsolo Á, de la Torre B. Type 2 Diabetes Mellitus Associated with Obesity (Diabesity). The Central Role of Gut Microbiota and Its Translational Applications. Nutrients. 2020; 12-2749
11. Castro-Barquero S, Lamuela-Raventós RM, Doménech M, Estruch R. Relationship between Mediterranean Dietary Polyphenol Intake and Obesity. Nutrients 2018; 10:1523.
12. Roberfroid M, Gibson G R, Hoyles L, McCartney A L, Rastall R, Rowland I, Wolvers D, Watzl B, Szajewska H, Stahl B, et al. Prebiotic effects: Metabolic and health benefits. Br. J. Nutr. 2010; 104:S1-S63
13. Abraham B P, Quigley E M M. Probiotics in Inflammatory Bowel Disease. Gastroenterol Clin North Am. 2017; 46:769-782.
14. Asemi Z, Samimi M, Tabassi Z, et al. Effect of daily consumption of probiotic yoghurt on insulin resistance in pregnant women: a randomized controlled trial. Eur J Clin Nutr. 2013; 67:71-74
15. Wilkins T, Sequoia J. Probiotics for Gastrointestinal Conditions: A Summary of the Evidence. Am Fam Physician. 2017; 96:170-178.
16. Matthews D R, Hosker J P, Rudenski A S, Naylor B A, Treacher D F, Turner R C. Homeostasis model assessment: insulin resistance and ß-cell function from fasting plasma glucose and insulin concentrations in man. Diabetologia 1985; 28:412-419
17. Pfützner A., Kunt T., Mondok A., Pahler S., Konrad T., Luebben G., Forst T.: Fasting Intact Proinsulin is a Highly Specific Predictor of Insulin Resistance in Type 2 Diabetes. Diabetes Care 2004; 27:682-687
18. Pfützner A., Kann P., Pfutzner A. H., Kunt T., Larbig M., Weber M. M., Forst T.: Intact and total proinsulin: new aspects for diagnosis and treatment of type 2 diabetes. Clin. Lab. 2004; 50:567-573
19. Schöndorf T., Maiworm A., Emisson N., Forst T., Pfützner A.: Biological Background and Role of Adiponectin as Marker for Insulin Resistance and Cardiovascular Risk. Clin Lab. 2005; 51:489-494
20. Pfützner A., Forst T.: HsCRP as cardiovascular risk marker in patients with diabetes mellitus. Diab. Technol. Ther. 2006; 8:28-36
21. International Diabetes Federation. IDF Diabetes Atlas 2019 https-J/www.diabetesatgas.org
22. Cree-Green M, Triolo T M, Nadeau K J. Etiology of insulin resistance in youth with type 2 diabetes. Curr Diab Rep. 2013; 13:81-88.
23. Mizokami-Stout K, Cree-Green M, Nadeau KJ. Insulin resistance in type 2 diabetic youth. Curr Opin Endocrinol Diabetes Obes. 2012; 19-255-262.
24. American Diabetes Association. Pharmacologic Approaches to Glycemic Treatment: standards of Medical Care in Diabetes-2021. Diabetes Care. 2021; 44(Suppl. 1):S111-S124
25. Davies M J, D'Alessio D A, Fradkin J, Keman W N, Mathieu C, Mingrone G, Rossing P, Tsapas A, Wexler D J, Buse J B. Management of Hyperglycemia in Type 2 Diabetes, 2018. A Consensus Report by the American Diabetes Association (ADA) and the European Association for the Study of Diabetes (EASD). Diabetes Care. 2018; 41:2669-2701
26. Qin J, Li Y, Cai Z, et al. A metagenome-wide association study of gut microbiota in type 2 diabetes. Nature. 2012; 490:55-60.
27. Larsen N, Vogensen F K, van den Berg F W, et al. Gut microbiota in human adults with type 2 diabetes differs from non-diabetic adults. PLoS One. 2010; 5:e9085. doi: 10.1371fjournal.pone.0009085.
28. Sato J, Kanazawa A, Ikeda F, et al. Gut dysbiosis and detection of “live gut bacteria” in blood of Japanese patients with type 2 diabetes. Diabetes Care. 2014; 37-2343-2350.
29. Soyucen E, Gulcan A, Aktuglu-Zeybek A C, et al. Differences in the gut microbiota of healthy children and those with type 1 diabetes. Pediatr Int. 2014; 56:336-343.
30. Bekkering P, Jafri I, van Overveld F J, et al. The intricate association between gut microbiota and development of type 1, type 2 and type 3 diabetes. Expert Rev Clin Immunol. 2013; 9:1031-1041.
31. Everard A, Cani P D. Diabetes, obesity and gut microbiota. Best Pract Res Clin Gastroenterol. 2013; 27:73-83.
32. Everard A, Lazarevic V, Derrien M, et al. Responses of gut microbiota and glucose and lipid metabolism to prebiotics in genetic obese and diet-induced leptin-resistant mice. Diabetes. 2011; 60:2775-2786.
33. Lin C H, Lin C C, Shibu M A, et al. Oral Lactobacillus reuteri GMN-32 treatment reduces blood glucose concentrations and promotes cardiac function in rats with streptozotocin-induced diabetes mellitus. Br J Nutr. 2014; 111:598-605.
34. Yadav H, Jain S, Sinha P R. Effect of skim milk and dahi (yogurt) on blood glucose, insulin, and lipid profile in rats fed with high fructose diet. J Med Food. 2006; 9:328-335.
35. Andreasen A S, Larsen N, Pedersen-Skovsgaard T, et al. Effects of Lactobacillus acidophilus NCFM on insulin sensitivity and the systemic inflammatory response in human subjects. Br J Nutr. 2010; 104:1831-1838.
36. Shaken H, Hadaegh H, Abedi F, et al. Consumption of synbiotic bread decreases triacylglycerol and VLDL levels while increasing HDL levels in serum from patients with type-2 diabetes. Lipids. 2014; 49:695-701.
37. Akbari V, Hendijani F, Effects of probiotic supplementation in patients with type 2 diabetes: systematic review and meta-analysis. Nutrition reviews, 2016; 74:274-284
38. Pfützner A., Weber M.M., Forst T.: A Biomarker Concept for Assessment of Insulin resistance, ß-Cell Function and Chronic Systemic Inflammation in Type 2 Diabetes mellitus. Clin Lab 2008; 54:485-490
39. Hollander P. Safety profile of acarbose, an alpha-glucosidase inhibitor. Drugs. 1992; Suppl 3:47-53.
40. Sels J P, Verdonk H E, Wolffenbuttel B H. Effects of acarbose (Glucobay) in persons with type 1 diabetes: a multicentre study. Diabetes Res Olin Pract. 1998 August; 41(2):139-45.
41. Kayibanda J F, Girouard C, Grégoire J P, Demers E, Moisan. Adherence to the evidence-based heart failure drug treatment: Are there sex-specific differences among new users?J. Res Social Adm Pharm. 2018; 146915-920

TABLE 1
Patient characteristics in both treatment arms

| | AB001 | Placebo |
|---|---|---|
| N | 20 | 20 |
| Gender f/m | 2/18 | 7/13 |
| Age [years] | 65.5 ± 8.8 | 67.5 ± 9.6 |
| HbA1c [%/]/[mmol/mol] | 7.4 ± 1.0/57 ± 11 | 7.1 ± 0.9/54 ± 10 |
| Diabetes treatment: | | |
| Diet only | 0 (0%) | 1 (5%) |
| Metformin | 11 (55%) | 9 (45%) |
| Sulfonylurea | 0 (0%) | 1 (5%) |
| DPPIV-inhibitor | 4 (20%) | 3 (15%) |
| Glitazone | 3 (15%) | 2 (10%) |
| SGLT-2 inhibitor | 4 (20%) | 8 (40%) |
| GLP-1 agonis | 4 (20%) | 5 (25%) |
| Insulin | 15 (75%) | 14 (70%) |

TABLE 2
Results for the glycemic and cardiodiabetes
parameters at baseline and endpoint

| | AB001 | | Placebo | |
|---|---|---|---|---|
| HbA1c | 7.4 ± 1.0 | 7.1 ± 0.9(*) | 7.0 ± 0.9 | 7.1 ± 0.8 |
| hsCRP | 3.6 ± 4.6 | 2.5 ± 2.4 | 3.0 ± 3.9 | 3.8 ± 3.8 |
| Adipo- | 9.3 ± 10.6 | 10.0 ± 10.7(*) | 7.2 ± 4.4 | 6.6 ± 3.7* |
| nectin | | | | |
| Intact | 9.0 ± 9.4 | 5.6 ± 4.6* | 6.2 ± 5.3 | 6.1 ± 6.0 |
| Proinsulin | | | | |
| Glucose | 160 ± 57 | 138 ± 45* | 138 ± 40 | 149 ± 44* |
| Insulin | 16.9 ± 14.8 | 14.1 ± 9.9(*) | 15.7 ± 14.7 | 17.9 ± 15.3* |
| HOMA-IR | 6.9 ± 6.8 | 5.1 ± 4.0* | 5.6 ± 5.3 | 6.8 ± 6.6* |
| Triglyc- | 204 ± 156 | 178 ± 148* | 162 ± 87 | 176 ± 92 |
| erides | | | | |
| Choles- | 177 ± 46 | 176 ± 47(*) | 169 ± 37 | 175 ± 35(*) |
| terol | | | | |
| LDL | 106 ± 39 | 107 ± 43 | 101 ± 35 | 110 ± 35* |
| cholesterol | | | | |
| HDL | 45 ± 14 | 48 ± 16(*) | 49 ± 17 | 47 ± 17 |
| cholesterol | | | | |
| Uric acid | 5.7 ± 0.7 | 5.8 ± 1.2 | 5.5 ± 1.3 | 5.8 ± 1.3 |

p vs. baseline:
*p < 0.05,
**: p < 0.01,
(*)strong trend = p < 0.1

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A composition comprising one or more bacterial species of the Bacillus genus, rice bran, L-cysteine and a high molecular weight low osmolality carbohydrate for use in treating and/or preventing type 2 diabetes, pre-diabetes, and/or a symptom thereof in a subject.

2. (canceled)

3. (canceled)

4. The composition for use according to claim 1, wherein administration of the composition to the subject:
(a) reduces the level of one or more of HbA1c, glucose, insulin, intact pro-insulin and/or hsCRP in the blood of the subject;
(b) reduces the HOMA-IR score of the subject; and/or
(c) increases the level of adiponectin in the blood of the subject.

5. (canceled)

6. The composition for use according to claim 4, wherein administration of the composition to the subject degrades glucose in the subject and/or metabolises glucose in the gut of the subject.

7. (canceled)

8. The composition for use according to claim 4, wherein administration of the composition to the subject reduces absorption of glucose into the blood of the subject.

9. (canceled)

10. The composition for use or method, according to claim 1, wherein the one or more bacterial species of the Bacillus genus is selected from at least one of: B. subtilis and B. coagulans.

11. The composition for use according to claim 10, wherein the composition comprises B. subtilis and B. coagulans.

12. The composition for use according to claim 11, wherein the B. subtilis species is selected from the group consisting of: Bacillus subtilis strain DFM 0326 (LMG P-32899) and Bacillus subtilis strain DFM 1015 (LMG P-32900).

13. The composition for use according to claim 1, wherein the high molecular weight low osmolality carbohydrate is dextrin.

14. The composition for use according to claim 1, wherein the composition further comprises one or more bacterial species selected from the group consisting of: Bacillus amyloliquefaciens, Bacillus velezensis, Bacillus sp MT 03, Bacillus atrophaeus, and Pediococcus pentosaceus.

15. The composition for use according to claim 1, wherein the composition comprises at least about 20% w/w of L-cysteine.

16. The composition for use according to claim 1, wherein the composition comprises at least about 1×10⁵ of bacteria of the Bacillus genus.

17. The composition for use according to claim 1, wherein the composition comprises at least about 73% w/w of rice bran.

18. The composition for use according to claim 1, wherein the composition comprises at least about 0.5% w/w of high molecular weight low osmolality carbohydrate.

19. The composition for use according to claim 1, wherein the composition further comprises one or more of: vitamin B12, a fatty acid magnesium salt, calcium phosphate, potassium phosphate, silicon dioxide and cellulose.

20. The composition for use according to claim 1, wherein the composition is formulated as an acid resistant tablet or capsule having a film coating which comprises hydroxypropyl methylcellulose (HPMC).

21. (canceled)

22. The composition for use according to claim 1, wherein the one or more bacterial species of the Bacillus genus is not genetically modified.

23. The composition for use according to claim 4, wherein the composition is for daily administration.

24. (canceled)

25. (canceled)

26. (canceled)

27. (canceled)

28. The composition for use according to claim 6 wherein administration of the composition to the subject metabolises glucose in the intestine of the subject.

29. The composition for use according to claim 12 wherein the B. coagulans species is Bacillus coagulans strain DFM 0705 (LMG P-32921).

30. The composition for use according to claim 19 wherein the fatty acid magnesium salt is magnesium stearate.
